SEQUENCE LISTING (37 sequences)

SEQ ID NO: 1 - DNA, 1995 bp, Rhodococcus sp.
atggccacctacctcgtcaccggcggaaccggcttcctgggtcggcacgtgctgcccctgatcctcgaccgcgatgaatcagccgagatccacgtcctcgtccggcgcgcgtccgtcgcgcggtgggaggcgctcgtcgacggcatgccgggcggcgaacgcgtgcatccgctgatcggcgacctgaccgagcccggcctcgcactcacctcggcgccggcggccgaccacgtcctgcacctcggcgcgatctacgacctcacggcgggcgacgaacaggccaccacgaacgtcgacggcacccgctcggtgatcgcactggcccgggaattggatgcgacgctgcaccacgtctcgtccatcgccgtcgcgggggatcacgaaggtccgttcagggaaaccgatttcgatctcgggcagggtttccccaccccgtatcaccgcaccaagttcgaggcggagaagctggtccgggaagccgacggcctgcggtggcgcatctaccgcccctccgcggtggtcggcagttcggtcaccggcgagatggacaagatcgacggcccgtactacctcttccccgccatcgcggcgctcgcgaaactccccgcggccctgcccgtggccgtcccgcggatcggatcgaccaacgtcgtccccgtcgactacgtggccgcggccatcgtggagttgatgtctgcaccgggacgggacggccggacgttccatctcgtcaatccgcgcccccagccggtccgcgagatctacgccgcgctcgccgaggccgccggtgcaccgcatcccgcggccggactacccggcggactcgtccgccccttcctcactcccacccccgactcggccctcgacacgggccgcaaagtggtgctcggcgccctcggcatcccgcccgtgctcgtcgagaacatgacactgcccacggtgttcgtgaacgacgacacccgggaggcgctgcgcggcacaggcatcgacgtcccgcggttcgcgtcgtacgccgacaggctgtggtcgtactggcgcgagaacctcgacccggaccgagcgcgcaggccccacccggccggaccgctcgtgggcaggcacgtggtgatcaccggggcgtcgtccggcatcggccggagttccgcgatcgcgacggcccgcaagggcgcgaaggtcattctcctcgcccggcgcaccgaggaactcgacgccgtggtggccgagatccgcgcggcgggcggcgacgcctacggctacccctgcgacatcaccgacgacgagtcggtcgagcacaccgtcaaagccgttctcgccgaacacgatcacgtcgacatgctcgtcaacaacgccggacggtcgatccggcggggcctctacaactccacggaccggctccacgatttcgagcggaccatggccgtcaactacttcggcgcggtccggctcgtcctcgccttcctcccgcagatgcgcgaccggcggttcgggcacatcgtcaacatcagcagcgccggggtccagggcgcgacgccgcggttcgccgcctacgtcgccagcaagtcggccctcgacggcttcactgacgtcgcggcgtccgaaaccctctcggacggaatcacgttcaccaccatccacatgccactcgtcgagacgccgatgatcgcgcccaccggccggtacaacatcggaccgatcacctccccggagaaggcggcggcaatggtcgtccgggcgctgatcgaacgccccaagcgcatcgacgtcccgaccggcaccctcggggaactcggccacgtcttcgcacccgccgtcaaggatcgcgtgatgcaccaaatgtatcgcatgttccccgattccccggcggcgaaggggcagagcacggacgacgcggcgccggcacctcagcacacaccggaacccaggcaccacacgtcgtcggtgctcggccgcgcggcccggaaggtcggaaggctggtgcccggcacccactggtga

SEQ ID NO: 2 - protein, 664 aa, Rhodococcus sp.
MATYLVTGGTGFLGRHVLPLILDRDESAEIHVLVRRASVARWEALVDGMPGGERVHPLIGDLTEPGLALTSAPAADHVLHLGAIYDLTAGDEQATTNVDGTRSVIALARELDATLHHVSSIAVAGDHEGPFRETDFDLGQGFPTPYHRTKFEAEKLVREADGLRWRIYRPSAVVGSSVTGEMDKIDGPYYLFPAIAALAKLPAALPVAVPRIGSTNVVPVDYVAAAIVELMSAPGRDGRTFHLVNPRPQPVREIYAALAEAAGAPHPAAGLPGGLVRPFLTPTPDSALDTGRKVVLGALGIPPVLVENMTLPTVFVNDDTREALRGTGIDVPRFASYADRLWSYWRENLDPDRARRPHPAGPLVGRHVVITGASSGIGRSSAIATARKGAKVILLARRTEELDAVVAEIRAAGGDAYGYPCDITDDESVEHTVKAVLAEHDHVDMLVNNAGRSIRRGLYNSTDRLHDFERTMAVNYFGAVRLVLAFLPQMRDRRFGHIVNISSAGVQGATPRFAAYVASKSALDGFTDVAASETLSDGITFTTIHMPLVETPMIAPTGRYNIGPITSPEKAAAMVVRALIERPKRIDVPTGTLGELGHVFAPAVKDRVMHQMYRMFPDSPAAKGQSTDDAAPAPQHTPEPRHHTSSVLGRAARKVGRLVPGTHW

SEQ ID NO: 3 - DNA, 1368 bp, Rhodococcus sp.
atgacgcccctgaatcccactgaccagctctttctctggctggaaaaacgccagcagcccatgcatgtgggcggcctccagctgttttccttccccgaaggcgcgccggacgactatgtcgcgcagctggcagaccagcttcggcagaagacggaggtgaccgccccctttaaccagcgcctgagctatcgcctgggccagccggtatgggtggaggatgagcacctggaccttgagcatcatttccgcttcgaggcgctgcccacacccgggcgtattcgggagctgctgtcgttcgtatcggcggagcattcgcacctgatggaccgggagcgccccatgtgggaggtgcacctgatcgagggcctgaaagaccggcagtttgcgctctacaccaaggttcaccattccctggtggacggtgtctcggccatgcgcatggccacccggatgctgagtgaaaacccggacgaacacggcatgccgccaatctgggatctgccttgcctgtcacgggataggggtgagtcggacggacactccctctggcgcagtgtcacccatttgctggggctttcggaccgccagctcggcaccattcccactgtggcaaaggagctactgaaaaccatcaatcaggcccggaaggatccggcctacgactccattttccatgccccgcgctgcatgctgaaccagaaaatcaccggttcccgtcgattcgccgctcagtcctggtgcctgaaacggattcgcgccgtatgcgaggcctacggcaccacggtcaacgatgtcgtgactgccatgtgcgcagcggctctgcgtacctatctgatgaatcaggatgccttgccggagaaaccactggtggcctttgtgccggtgtcgctacgccgggacgacagctccggcggcaaccaggtaggcgtcatcctggcgagccttcacaccgatgtgcaggacgccggcgaacgactgttaaaaattcaccacggcatggaagaggccaagcagcgctaccggcatatgagcccggaggaaatcgtcaactacacggccctgaccctggcgccggccgccttccacctgctgaccgggctggcgcccaagtggcagaccttcaatgtggtgatttccaatgtccccgggccatccaggcccctgtactggaacggggcgaaactggaaggcatgtatccggtgtctatcgatatggacaggctggccctgaacatgacactgaccagctataacgaccaggtggagttcggcctgattggctgtcgccggaccctgcccagcctgcaacggatgctggactacctggaacagggtctggcagagctggagctcaacgccggtctgtaa

SEQ ID NO: 4 - protein, 455 aa, Rhodococcus sp.
MTPLNPTDQLFLWLEKRQQPMHVGGLQLFSFPEGAPDDYVAQLADQLRQKTEVTAPFNQRLSYRLGQPVWVEDEHLDLEHHFRFEALPTPGRIRELLSFVSAEHSHLMDRERPMWEVHLIEGLKDRQFALYTKVHHSLVDGVSAMRMATRMLSENPDEHGMPPIWDLPCLSRDRGESDGHSLWRSVTHLLGLSDRQLGTIPTVAKELLKTINQARKDPAYDSIFHAPRCMLNQKITGSRRFAAQSWCLKRIRAVCEAYGTTVNDVVTAMCAAALRTYLMNQDALPEKPLVAFVPVSLRRDDSSGGNQVGVILASLHTDVQDAGERLLKIHHGMEEAKQRYRHMSPEEIVNYTALTLAPAAFHLLTGLAPKWQTFNVVISNVPGPSRPLYWNGAKLEGMYPVSIDMDRLALNMTLTSYNDQVEFGLIGCRRTLPSLQRMLDYLEQGLAELELNAGL

SEQ ID NO: 5 - DNA, 1422 bp, Rhodococcus sp.
atgaaacgtctcggaaccctggacgcctcctggctggcggttgaatctgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgataaggatatcgatctggattatcacgtccggcactcagccctgcctcgccccggcggggagcgcgaactgggtattctggtatcccgactgcactctaaccccctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacggtacgcccacaccaacgccgtggtgcaaaaaccgacaaagaggccagcgtgcccgcagcggtttcccaggccatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgtgctttacctgtgtggcaccgcattgcggcgctttctggctgagcagaacaatctgccagacaccccgctgacggctggtataccggtgaatatccggccggcagacgacgagggtacgggcacccagatcagtttcatgattgcctcgctggccaccgacgaagctgatccgttgaaccgcctgcaacagatcaaaacctcgacccgacgggccaaggagcacctgcagaaacttccaaaaagtgccctgacccagtacaccatgctgctgatgtcaccctacattctgcaattgatgtcaggtctcggggggaggatgcgaccagtcttcaacgtgaccatttccaacgtgcccggcccggaaggcacgctgtattatgaaggagcccggcttgaggccatgtatccggtatcgctaatcgctcacggcggcgccctgaacatcacctgcctgagctatgccggatcgctgaatttcggttttaccggctgtcgggatacgctgccgagcatgcagaaactggcggtttataccggtgaagctctggatgagctggaatcgctgattctgccacccaagaagcgcgcccgaacccgcaagtaa

SEQ ID NO: 6 - protein, 473 aa, Rhodococcus sp.
MKRLGTLDASWLAVESEDTPMHVGTLQIFSLPEGAPETFLRDMVTRMKEAGDVAPPWGYKLAWSGFLGRVIAPAWKVDKDIDLDYHVRHSALPRPGGERELGILVSRLHSNPLDFSRPLWECHVIEGLENNRFALYTKMHHSMIDGISGVRLMQRVLTTDPERCNMPPPWTVRPHQRRGAKTDKEASVPAAVSQAMDALKLQADMAPRLWQAGNRLVHSVRHPEDGLTAPFTGPVSVLNHRVTAQRRFATQHYQLDRLKNLAHASGGSLNDIVLYLCGTALRRFLAEQNNLPDTPLTAGIPVNIRPADDEGTGTQISFMIASLATDEADPLNRLQQIKTSTRRAKEHLQKLPKSALTQYTMLLMSPYILQLMSGLGGRMRPVFNVTISNVPGPEGTLYYEGARLEAMYPVSLIAHGGALNITCLSYAGSLNFGFTGCRDTLPSMQKLAVYTGEALDELESLILPPKKRARTRK

SEQ ID NO: 7 - protein, 458 aa, Rhodococcus sp.
MRPLHPIDFIFLSLEKRQQPMHVGGLFLFQIPDNAPDTFIQDLVNDIRISKSIPVPPFNNKLNGLFWDEDEEFDLDHHFRHIALPHPGRIRELLIYISQEHSTLLDRAKPLWTCNIIEGIEGNRFAMYFKIHHAMVDGVAGMRLIEKSLSHDVTEKSIVPPWCVEGKRAKRLREPKTGKIKKIMSGIKSQLQATPTVIQELSQTVFKDIGRNPDHVSSFQAPCSILNQRVSSSRRFAAQSFDLDRFRNIAKSLNVTINDVVLAVCSGALRAYLMSHNSLPSKPLIAMVPASIRNDDSDVSNRITMILANLATHKDDPLQRLEIIRRSVQNSKQRFKRMTSDQILNYSAVVYGPAGLNIISGMMPKRQAFNLVISNVPGPREPLYWNGAKLDALYPASIVLDGQALNITMTSYLDKLEVGLIACRNALPRMQNLLTHLEEEIQLFEGVIAKQEDIKTAN

SEQ ID NO: 8 - protein, 451 aa, Rhodococcus sp.
MARKLSIMDSGWLMMETRETPMHVGGLALFAIPEGAPEDYVESIYRYLVDVDSICRPFNQKIQSHLPLYLDATWVEDKNFDIDYHVRHSALPRPGRVRELLALVSRLHAQRLDPSRPLWESYLIEGLEGNRFALYTKMHHSMVDGVAGMHLMQSRLATCAEDRLPAPWSGEWDAEKKPRKSRGAAAANAGMKGTMNNLRRGGGQLVDLLRQPKDGNVKTIYRAPKTQLNRRVTGARRFAAQSWSLSRIKAAGKQHGGTVNDIFLAMCGGALRRYLLSQDALSDQPLVAQVPVALRSADQAGEGGNAITTVQVSLGTHIAQPLNRLAAIQDSMKAVKSRLGDMQKSEIDVYTVLTNMPLSLGQVTGLSGRVSPMFNLVISNVPGPKETLHLNGAEMLATYPVSLVLHGYALNITVVSYKNSLEFGVIGCRDTLPHIQRFLVYLEESLVELEP

SEQ ID NO: 9 - protein, 661 aa, Marinobacter sp.
MNYFLTGGTGFIGRFLVEKLLARGGTVYVLVREQSQDKLERLRERWGADDKQVKAVIGDLTSKNLGIDAKTLKSLKGNIDHVFHLAAVYDMGADEEAQAATNIEGTRAAVQAAEAMGAKHFHHVSSIAAAGLFKGIFREDMFEEAEKLDHPYLRTKHESEKVVREECKVPFRIYRPGMVIGHSETGEMDKVDGPYYFFKMIQKIRHALPQWVPTIGIEGGRLNIVPVDFVVDALDHIAHLEGEDGNCFHLVDSDPYKVGEILNIFCEAGHAPRMGMRIDSRMFGFIPPFIRQSIKNLPPVKRITGALLDDMGIPPSVMSFINYPTRFDTRELERVLKGTDIEVPRLPSYAPVIWDYWERNLDPDLFKDRTLKGTVEGKVCVVTGATSGIGLATAEKLAEAGAILVIGARTKETLDEVAASLEAKGGNVHAYQCDFSDMDDCDRFVKTVLDNHGHVDVLVNNAGRSIRRSLALSFDRFHDFERTMQLNYFGSVRLIMGFAPAMLERRRGHVVNISSIGVLTNAPRFSAYVSSKSALDAFSRCAAAEWSDRNVTFTTINMPLVKTPMIAPTKIYDSVPTLTPDEAAQMVADAIVYRPKRIATRLGVFAQVLHALAPKMGEIIMNTGYRMFPDSPAAAGSKSGEKPKVSTEQVAFAAIMRGIYW

SEQ ID NO: 10 - protein, 513 aa, Marinobacter sp.
MAIQQVHHADTSSSKVLGQLRGKRVLITGTTGFLGKVVLERLIRAVPDIGAIYLLIRGNKRHPDARSRFLEEIATSSVFDRLREADSEGFDAFLEERIHCVTGEVTEAGFGIGQEDYRKLATELDAVINSAASVNFREELDKALAINTLCLRNIAGMVDLNPKLAVLQVSTCYVNGMNSGQVTESVIKPAGEAVPRSPDGFYEIEELVRLLQDKIEDVQARYSGKVLERKLVDLGIREANRYGWSDTYTFTKWLGEQLLMKALNGRTLTILRPSIIESALEEPAPGWIEGVKVADAIILAYAREKVTLFPGKRSGIIDVIPVDLVANSIILSLAEALGEPGRRRIYQCCSGGGNPISLGEFIDHLMAESKANYAAYDHLFYRQPSKPFLAVNRALFDLVISGVRLPLSLTDRVLKLLGNSRDLKMLRNLDTTQSLATIFGFYTAPDYIFRNDELMALANRMGEVDKGLFPVDARLIDWELYLRKIHLAGLNRYALKERKVYSLKTARQRKKAA

SEQ ID NO: 11 - protein, 295 aa, Acinetobacter sp.
MNKKLEALFRENVKGKVALITGASSGIGLTIAKRIAAAGAHVLLVARTQETLEEVKAAIEQQGGQASIFPCDLTDMNAIDQLSQQIMASVDHVDFLINNAGRSIRRAVHESFDRFHDFERTMQLNYFGAVRLVLNLLPHMIKRKNGQIINISSIGVLANATRFSAYVASKAALDAFSRCLSAEVLKHKISITSIYMPLVRTPMIAPTKIYKYVPTLSPEEAADLIVYAIVKRPKRIATHLGRLASITYAIAPDINNILMSIGFNLFPSSTAALGEQEKLNLLQRAYARLFPGEHW

SEQ ID NO: 12 - protein, 650 aa, Mycobacterium sp.
MRYVVTGGTGFIGRHVVSRLLDGRPEARLWALVRRQSLSRFERLAGQWGDRVRPLVGDLTELELSERTIAELGDIDHVLHCAAVHDTTWADATRAVIELAARLDATFHHVSSIAVAGDFAGHYTEADFDVGQRLPTPYHRMTFEAERLVRSTPGLRYRIYRPAVVVGDSRTGEMDTIDGPYYLFGVLAKLAVLPSFTPMLLPDIGRTNIVPVDYVADALVALMHADGRDGQTFHLTAPTAIGLRGIYRGIAGAAGLPPLLGTLPGFVAAPVLNARGRAKVLRNMAATQLGIPAEIFDVVGCAPTFTSDTTREALRGTGIHVPEFATYAPGLWRYWAEHLDPDRARRNDPLLGRHVIITGASSGIGRASAIAVAKRGATVFALARNGNALDELVTEIRAHGGQAHAFTCDVTDSASVEHTVKDILGRFDHVDYLVNNAGRSIRRSVVNSTDRLHDYERVMAVNYFGAVRMVLALLPHWRERRFGHVVNVSSAGVQARNPKYSSYLPTKAALDAFADVVASETLSDHITFTNIHMPLVATPMIVPSRRLNPVRAISAERAAAMVIRGLVEKPARIDTPLGTLAEAGNYVAPRLSRRILHQLYLGYPDSAAAQGISRPDADRPPAPRRPRRSARAGVPRPLRRLGRLVPGVHW

SEQ ID NO: 13 - protein, 341 aa, Mycobacterium sp.
MNLGDLTNFVEKPLAAVSNIVNTPNSAGRYRPFYLRNLLDAVQGRNLNDAVKGKVVLITGGSSGIGAAAAKKIAEAGGTVVLVARTLENLENVANDIRAIRGNGGTAHVYPCDLSDMDAIAVMADQVLGDLGGVDILINNAGRSIRRSLELSYDRIHDYQRTMQLNYLGAVQLILKFIPGMRERHFGHIVNVSSVGVQTRAPRFGAYIASKAALDSLCDALQAETVHDNVRFTTVHMALVRTPMISPTTIYDKFPTLTPDQAAGVITDAIVHRPRRASSPFGQFAAVADAVNPAVMDRVRNRAFNMFGDSSAAKGSESQTDTSELDKRSETFVRATRGIHW

SEQ ID NO: 14 - DNA, 39 bp, Artificial Sequence
  Primer
ggtcgactctagagcggccgctcactcttctgctcggcc

SEQ ID NO: 15 - DNA, 57 bp, Artificial Sequence
  Primer
aacaaaattatttctagacgatatcgccgtccattatacctcctcacgtgacgtgag

SEQ ID NO: 16 - DNA, 42 bp, Artificial Sequence
  Primer
tttaagaaggagatatacatatggccacctacctcgtcaccg

SEQ ID NO: 17 - DNA, 78 bp, Artificial Sequence
  Primer
atggtgatggtgatgctcgaggctgctgccctggaaatacaagttttctctgccgctgctccagtgggtgccgggcac

SEQ ID NO: 18 - DNA, 41 bp, Artificial Sequence
  Primer
tttaagaaggagatatacatatggccacctacctcgtcacc

SEQ ID NO: 19 - DNA, 37 bp, Artificial Sequence
  Primer
gtgccggtgggtcgactagtcaccagtgggtgccggg

SEQ ID NO: 20 - DNA, 28 bp, Artificial Sequence
  Primer
tgaggtgcgaggacgtggatctcggctg

SEQ ID NO: 21 - DNA, 38 bp, Artificial Sequence
  Primer
cgggtaccgagctcgaattcgtctccgcgccatcacgc

SEQ ID NO: 22 - DNA, 39 bp, Artificial Sequence
  Primer
gctatgacatgattacgaattcggccggggtgcgggtca

SEQ ID NO: 23 - DNA, 32 bp, Artificial Sequence
  Primer
acgtcctcgcacctcagcacacaccggaaccc

SEQ ID NO: 24 - DNA, 39 bp, Artificial Sequence
  Primer
ctttaagaaggagatatacatatggtgagcaagggcgag

SEQ ID NO: 25 - DNA, 46 bp, Artificial Sequence
  Primer
cacgggtgccggtgggtcgactagtcacttgtacagctcgtccatg

SEQ ID NO: 26 - DNA, 73 bp, Artificial Sequence
  Primer
ggctgcaggtcgactctagagcggccgcatcgatcattgatatcgtctagaaataattttgtttaactttaag

SEQ ID NO: 27 - DNA, 38 bp, Artificial Sequence
  Primer
gcgttggccgattcattaatactagagtcccgctgagg

SEQ ID NO: 28 - DNA, 39 bp, Artificial Sequence; identical to SEQ ID NO: 14
  Primer

SEQ ID NO: 29 - DNA, 57 bp, Artificial Sequence; identical to SEQ ID NO: 15
  Primer

SEQ ID NO: 30 - DNA, 41 bp, Artificial Sequence; identical to SEQ ID NO: 18
  Primer

SEQ ID NO: 31 - DNA, 37 bp, Artificial Sequence; identical to SEQ ID NO: 19
  Primer

SEQ ID NO: 32 - DNA, 39 bp, Artificial Sequence
  Primer
ggtcgactctagagcggccgcaccgctctggtcagcgac

SEQ ID NO: 33 - DNA, 42 bp, Artificial Sequence
  Primer
aacaaaattatttctagacgatatctggagtcggttgaccag

SEQ ID NO: 34 - DNA, 39 bp, Artificial Sequence; identical to SEQ ID NO: 32
  Primer

SEQ ID NO: 35 - DNA, 44 bp, Artificial Sequence
  Primer
aacaaaattatttctagacgatatccttgcgacgaaaggaactc

SEQ ID NO: 36 - DNA, 39 bp, Artificial Sequence; identical to SEQ ID NO: 32
  Primer

SEQ ID NO: 37 - DNA, 48 bp, Artificial Sequence
  Primer
aacaaaattatttctagacgatatcgaaaggaactctacaacagcgac

(19) United States
(12) Patent Application Publication
(10) Pub. No.: US 2020/0063169 A1
(43) Pub. Date: Feb. 27, 2020

(54) METHODS AND COMPOSITIONS FOR THE PRODUCTION OF LIPIDS

(71) Applicant: THE UNIVERSITY OF BRITISH COLUMBIA, Vancouver (CA)

(72) Inventors: Lindsay ELTIS, Vancouver (CA); James W. ROUND, Vancouver (CA); Raphael ROCCOR, Vancouver (CA)

(21) Appl. No.: 16/609,584

(22) PCT Filed: May 8, 2018
(86) PCT No.: PCT/IB2018/053201

§ 371 Date: Oct. 30, 2019

Related U.S. Application Data
(60) Provisional application No. 62/503,082, filed on May 8, 2017.

(51) Int. Cl. C12P 7/64; C12P 7/04; C12N 9/10

(52) U.S. Cl. CPC C12P 7/6436; C12P 7/04; C12N 15/63; C12Y 102/01084; C12N 9/1029

(57) ABSTRACT

The present invention relates to methods and compositions for the production of lipids. More specifically, the present inventions relates to methods and compositions for the production of fatty alcohols and/or wax esters.

### FIELD OF INVENTION

The present invention relates to methods and compositions for the production of lipids. More specifically, the present invention relates to methods and compositions for the production of fatty alcohols and/or wax esters.

### BACKGROUND OF THE INVENTION

The transformation of biomass into value-added products is at the core of the biotechnology industry. While the industry's early focus was on the use of organisms for the production of biofuels, the production of higher-value products, for example 1,4-butanediol or succinic acid, is gaining increased attention.

Many bacterial species, particularly the mycolic acid-producing Actinobacteria, synthesize neutral lipids for energy storage. For example, the soil bacterium Rhodococcus jostii RHA1 (RHA1 hereafter) accumulates neutral lipids up to 70% of cellular dry weight (CDW) in response to environmental stresses such as nitrogen limitation [1-3]. These neutral lipids may often be primarily triacylglycerides (TAGs), and may be stored in lipid bodies within the cytoplasm [4]. Proteins associated with these carbon storage organelles [5] may facilitate the dynamic shuffling of lipids between utilization and storage, allowing the bacterium to sequester intracellular energy reserves as needed.

Due in part to their remarkable biosynthetic capacity, there has been considerable interest in using oleaginous microorganisms to sustainably produce neutral lipids to replace oleochemicals currently derived from palm oils and petroleum [6-8]. Earlier research in rhodococci has focused on the production of TAGs to manufacture biodiesel [7]. For example, Holder et al. conducted a comparative analysis of the Rhodococcus opacus PD630 genome sequence with that of Rhodococcus jostii RHA1, Raistonia eutropa H16, and Corynebacterium glutamicum, and thereby identified a set of biofuel target genes for TAG production [9]. In WO 2010/147642 a method of producing TAGs from biomass specifically using Rhodococcus opacus PD630 is disclosed, while WO 2016/075623 teaches a process for the industrial production of lipids using native bacteria of the genus Rhodococcus to synthesize and accumulate TAGs. Already earlier, Alvarez et al. reported the identification of phenyldecanoic acid as a constituent of TAGs and wax esters produced by the native bacterium Rhodococcus opacus PD630 [10] and specifically reviewed the metabolism of TAGs in Rhodococcus species. The poor economics of biodiesel has however hindered the development and adoption of industrial-scale processes [11, 12].

An alternate approach may be to harness the biosynthetic potential of rhodococci to produce higher-value lipids. For example, bacterial-derived neutral lipids may be useful in the cosmetic and pharmaceuticals industries, as neutral lipids may play an important role in the health of skin [14, 15]. Furthermore, lipids have been implicated in the maintenance of a healthy skin microbiome [16, 17], which was found to have a profound effect on wound healing and healthy skin [18, 19]. Finally, the application of biological oils may speed wound healing and improve overall treatment outcomes [20-22]. Wax esters (WEs) are one of the characteristic components of human sebum, accounting for 26% of lipids found on the human skin [23]. Adult epidermis shows abundant unsaturated WEs with a medium chain length of 34 [24]. They act as protective barrier and may be involved in photoprotection, antimicrobial activity, delivery of fat-soluble anti-oxidants to the skin surface and pro- and anti-inflammatory activity [23, 25].

Found throughout many domains of life, WEs have both functional and structural roles [14, 26-28]. In some oleaginous bacteria, WEs appear to be primarily used to store energy [29, 30]. In these bacteria, WE synthesis may often involve the esterification of a fatty alcohol and an acyl-CoA in a reaction that may be catalyzed by a bifunctional WE synthase/acyl-CoA:diacylglycerol acyltransferase (WS/DGAT) [31, 32]. Under lipid-accumulating conditions, acyl-CoA substrates may be predominantly provided through lipid biosynthesis. When not available exogenously or generated as intermediates in the degradation of alkanes, fatty alcohols may be synthesized de novo from acyl-CoAs produced by fatty acid synthases [30, 33]. Some oleaginous Actinobacteria have been reported to produce WEs; for example, Mycobacterium tuberculosis accumulates WEs to ˜4% of total neutral lipids alongside TAGs during periods of stress [34], contributing to dormancy and antibiotic resistance [35]. Rhodococci also produce WEs under lipid-accumulating conditions when supplied with exogenous fatty alcohols or hydrocarbons [1]. Moreover, transient de novo production of WEs was reported in RHA1 by Barney et al [36]. Despite some teachings which reported that oleaginous rhodococci, such as RHA1 and Rhodococcus opacus PD630, harbored over a dozen WS/DGAT homologues, encoded by atf genes [1, 9] and that among the characterized rhodococcal WS/DGATs, Atf1_{PD630} had higher WS than DGAT activity [37], the capacity of rhodococci to produce WEs remained poorly understood.

### SUMMARY OF THE INVENTION

The present invention provides, in part, methods for producing fatty alcohols and wax esters.

In one aspect, there is provided a method for producing a fatty alcohol by: providing a host cell including a recombinant fatty acyl-coenzyme A reductase; and growing the host cell under conditions suitable for increased production of a fatty alcohol. In some embodiments, the recombinant fatty acyl-coenzyme A reductase may include a nucleic acid sequence substantially identical to SEQ ID NO. 1 or a fragment thereof. In some embodiments, the recombinant fatty acyl-coenzyme A reductase may include an amino acid sequence substantially identical to SEQ ID NO. 2 or a fragment thereof.

In another aspect, there is provided a method for producing a fatty alcohol by: providing a host cell, where the host cell is a Rhodococcus cell; and growing the host cell under conditions suitable for increased production of a fatty alcohol.

The host cell may further include a wax synthase. In some embodiments, the wax synthase may be recombinant or heterologous. In some embodiments, the wax synthase may include a nucleic acid sequence substantially identical to SEQ ID NOs. 3 or 5 or a fragment thereof. In some embodiments, the wax synthase may include an amino acid sequence substantially identical to SEQ ID NOs. 4 or 6 or a fragment thereof.

In some embodiments, the fatty alcohol may be converted into a wax ester within the host cell.

In some embodiments, the methods may include isolating the fatty alcohol or wax ester.

In another aspect, there is provided a fatty alcohol or wax ester produced in accordance with the methods described herein.

In another aspect, there is provided a vector including a polynucleotide including a sequence substantially identical to SEQ ID NO. 1 or a fragment thereof.

In some embodiments, the vector may further include a sequence substantially identical to SEQ ID NOs. 3 or 5 or a fragment thereof.

In some embodiments, the vector may be operably linked to a promoter, such as a constitutive promoter.

In another aspect, there is provided a host cell including a vector as described herein.

In some embodiments, the host cell may overproduce a fatty alcohol.

In some embodiments, the host cell may overproduce a wax ester.

In some embodiments, the host cell may overexpress a fatty acyl-coenzyme A reductase.

In some embodiments, the host cell may overexpress a wax synthase.

In another aspect, there is provided a kit including a vector or a host cell as described herein, in combination with instructions for producing a fatty alcohol or a wax ester.

In another aspect, there is provided a composition including a mixture of wax esters of twenty-eight to forty-five carbon atoms in length wherein at least one ester group is located at position C14, C15, C16, C17, C18, or C19 on the carbon chain. In some embodiments, the ester group of the wax esters may be located at position C14 in at least 0.5% of the mixture, at position C15 in at least 2% of the mixture, at position C16 in at least 10% of the mixture, at position C17 in at least 25% of the mixture, at position C18 in at least 20% of the mixture, at position C19 in at least 10%, and at position C20 in at least 3% of the mixture.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 is a schematic diagram showing bacterial WE biosynthesis pathways. Fatty alcohols can be generated through either a single enzyme (A) or consecutive reactions catalyzed by two enzymes (B). Fatty alcohols and an acyl-CoA are esterified to form WEs (C). The enzymes are: Fcr, alcohol-forming fatty acyl-CoA reductase; Acr, aldehyde-forming fatty acyl-CoA reductase; FaldR, fatty aldehyde reductase; WS/DGAT, WE synthase/acyl-CoA:diacylglycerol acyltransferase. RS30405 and RS09420 are the homologs identified in RHA1.

FIG. 2A is a GC/MS chromatogram of WE-containing lipid fraction in RHA1. Identified WEs are labeled according to total number of carbon atoms. Cells were grown on glucose and neutral lipids were extracted and fractionated using flash chromatography.

FIG. 2B is a mass spectrum of the detected C32 WEs in RHA1. Molecular ion and major fatty acyl fragments labeled. Cells were grown on glucose and neutral lipids were extracted and fractionated using flash chromatography.

FIG. 2C is a mass spectrum of WEs detected in RHA1 using the C16 RCOOH₂⁺ ion (m/z=257), the most abundant ion fragment associated with the WEs. Cells were grown on glucose and neutral lipids were extracted and fractionated using flash chromatography.

FIG. 3A shows the distribution of WE chain lengths detected in RHA1 under different conditions. Weighted mean chain lengths were calculated and are displayed above each column. Errors ranged from 0.01 to 0.17. Conditions were: C−, carbon-limited; N−, nitrogen limited; SNP, Sodium nitroprusside-treated RHA1; pTip-fcrA, RHA1 overproducing FcrA under N⁻ conditions. Sat. and Unsat. indicated saturated and unsaturated WEs/acyl-chains, respectively.

FIG. 3B shows the distribution of acyl-chain lengths of detected WEs in RHA1 under different conditions. Weighted mean chain lengths were calculated and are displayed above each column. Errors ranged from 0.01 to 0.17. Conditions were: C−, carbon-limited; N−, nitrogen limited; SNP, Sodium nitroprusside-treated RHA1; pTip-fcrA, RHA1 overproducing FcrA under N⁻ conditions. Sat. and Unsat. indicated saturated and unsaturated WEs/acyl-chains, respectively.

FIG. 4A shows 8 pg His₆-tagged FcrA purified from RHA1 and separated by SDS-PAGE.

FIG. 4B shows a GC/MS trace of a reaction mixture containing 1.4 μM FcrA-His₆, 100 μM oleoyl-CoA and 400 μM NADPH (20 mM Tris-HCl, pH 7.0, 50 mM NaCl) incubated for 20 hours.

FIG. 5A shows the WE content of RHA1 overproducing FcrA, total lipid extracts as visualized by TLC. Lanes were loaded with: 1, extract of RHA1 carrying an empty vector (pTipQC2); 2, extract of RHA1 overproducing FcrA (pTip-fcrA); and 3, stearyl stearate and glyceryl tripalmitate.

FIG. 5B shows the GC/MS analysis of WE fraction isolated from RHA1 overproducing FcrA from pTip-fcrA.

FIG. 6A shows the comparison of WEs in wild-type RHA1. Neutral lipids were extracted from exponentially growing cells in C⁻ conditions without sodium nitroprusside (SNP) treatment, fractionated on silica resin, and examined by GC/MS. The C16 RCOOH₂⁺ ion with an m/z of 257 was used to identify WEs. Similar trends were seen when other RCOOH₂⁺ ions were examined.

FIG. 6B shows the comparison of WEs in the RHA1 ΔfcrA mutant. Neutral lipids were extracted from exponentially growing cells in C⁻ conditions with sodium nitroprusside (SNP) treatment, fractionated on silica resin, and examined by GC/MS. The C16 RCOOH₂⁺ ion with an m/z of 257 was used to identify WEs. Similar trends were seen when other RCOOH₂⁺ ions were examined.

FIG. 7 shows WEs were produced from a RHA1 strain with genomically integrated fcrA expressed using a constitutive promoter. The empty integration vector pSET was used as a control. (WEs=wax esters, TAGs=triacylglycerides, FOHs=fatty alcohols).

FIG. 8 shows compositions of WEs in the engineered RHA1. Reported % of total WEs, by number of carbon atoms and level of desaturation.

FIG. 9 shows WE compositions of various sources. Even WEs contain an even number of C-atoms, while odd WEs contain an odd number of C-atoms.

FIG. 10 shows fatty alcohol composition of engineered RHA1. Reported as % of total fatty alcohols detected within WEs by number of carbons in the acyl-chain and the level of desaturation.

FIG. 11 shows fatty alcohol composition of the engineered RHA1. The presence of odd-chain fatty alcohols is noted.

FIG. 12 shows the nucleotide sequence of fcrA (SEQ ID NO: 1).

FIG. 13 shows the amino acid sequence of FcrA (SEQ ID NO: 2).

FIG. 14A shows PD630 overproducing FcrA accumulated WEs in both C− and N− conditions and separated by TLC

FIG. 14B shows WEs were 20 to 40% of total lipids in PD630.

FIG. 14C shows WEs were a mixture of saturated and unsaturated species in PD630.

FIG. 15 shows the composition of WEs isolated from PD630 overproducing FcrA.

FIG. 16 shows promoters of three different strengths, used to drive the expression of fcrA. WE accumulation depended on promoter strength.

FIG. 17A shows the use of the pTip expression plasmid to screen various wax synthases for their ability to increase WE accumulation in RHA1 carrying pSYN004-fcrA.

FIG. 17B shows that, under N-excess conditions (C− media, harvested in exponential), overproduction of WS1, WS2 or AtfA greatly increased WE accumulation.

FIG. 17C shows that, under N-limitation (N− media, harvested in stationary), overproduction of WS2 increased WE content˜8×versus the empty vector control.

FIG. 18 shows the nucleotide sequence of ws1 (SEQ ID NO: 3).

FIG. 19 shows the amino acid sequence of WS1 (SEQ ID NO: 4).

FIG. 20 shows the nucleotide sequence of ws2 (SEQ ID NO: 5).

FIG. 21 shows the amino acid sequence of WS2 (SEQ ID NO: 6).

FIG. 22 shows the amino acid sequence of AtfA (SEQ ID NO: 7).

FIG. 23 shows the amino acid sequence of AtfA2 (SEQ ID NO: 8).

FIG. 24 shows the amino acid sequence of Maqu_2507 (SEQ ID NO: 9).

FIG. 25 shows the amino acid sequence of Maqu_2220 (SEQ ID NO: 10).

FIG. 26 shows the amino acid sequence of Acr1 (SEQ ID NO: 11).

FIG. 27 shows the amino acid sequence of Rv3391 (SEQ ID NO: 12).

FIG. 28 shows the amino acid sequence of Rv1543 (SEQ ID NO: 13).

### DETAILED DESCRIPTION

The present disclosure provides, in part, methods and compositions for producing fatty alcohols and/or wax esters.

By a “fatty acid,” as used herein, is meant a molecule of the general chemical formula: R—COOH, where R is a chain of carbon atoms (the “chain length” or “carbon chain”). In some embodiments, the fatty acid may be a mixture of differing chain lengths.

By a “fatty alcohol” or “long-chain alcohol,” as used herein, is meant a molecule of the general chemical formula: R′—OH, where R′ is a carbon chain, as described herein. In some embodiments, the fatty alcohol may be a mixture of differing chain lengths.

By a “wax,” “wax ester,” or “WE,” as used herein, is meant an ester including a fatty acid and a fatty alcohol. In some embodiments, the WE may be of the general chemical formula RCOOR′, where R and R′ may independently be a carbon chain, as described herein. In some embodiments, the “acyl chain” of the WE may be the carbon chain donated by acyl-CoA (fatty acid). In some embodiments, the WE may include a mixture of WEs of differing chain lengths. In some embodiments, the WE may include a mixture of WEs as described herein. In some embodiments, the WE may include a mixture of WEs with at least one ester group located at C14, C15, C16, C17, C18, or C19 on the C-atom chain. In some embodiments, the WE may include a mixture of WEs of twenty-eight to forty-five C-atoms in length where the ester group may be located at C14 in at least 0.5%, at C15 in at least 2%, at C16 in at least 10%, at C17 in at least 25%, at C18 in at least 20%, at C19 in at least 10%, and at C20 in at least 3% of the WEs. In some embodiments, the WE may be a mixture similar to that of human sebum wax esters. In some embodiments, the WE may be a mixture similar to that of spermaceti wax esters.

In some embodiments, the fatty alcohol or WE composition may be similar to that derived from a natural source, such as a natural fat or oil. In some embodiments, the number of carbon atoms in the carbon chain may be any number produced by a natural source, such as a microorganism, plant or animal, or may be synthesized using laboratory techniques. In some embodiments, the carbon chain may be saturated or may be unsaturated. In some embodiments, the level of saturation of carbon chain of fatty alcohol or WE produced in accordance with the methods described herein may be different from that found in nature.

In some embodiments, the fatty acid, fatty alcohol or WE may include an even number of carbon atoms in the chain (R or R′). In some embodiments, the fatty acid, fatty alcohol or WE may include an odd number of carbon atoms in the chain. In some embodiments, the chain length of the fatty acid, fatty alcohol or WE may be any value from about 4 carbon atoms to about 45 carbon atoms, or about 28 carbon atoms to about 45 carbon atoms, or about 30 carbon atoms to about 38 carbon atoms, or about 30 carbon atoms to about 34 carbon atoms, or about 32 carbon atoms to about 36 carbon atoms, or about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 carbon atoms.

In some embodiments, the fatty acid, fatty alcohol or WE may be branched at any position of the carbon chain. In some embodiments, the carbon chain may be branched, for example, may include at least one C—C branch. In some embodiments, the carbon chain may include at least one C═C double-bond. In some embodiments, the branch may contain any value from about 1 to about 20 carbon atoms, such as 1, 2, 3, 4, 5, 6, 7, 8, 9, 10, 11, 12, 13, 14, 15, 16, 17, 18, 19, or 20 carbon atoms. In some embodiments, the carbon chain of the fatty acid, fatty alcohol or WE may be unbranched.

In some embodiments, the carbon chain may be optionally substituted, for example with one or more alkyl groups or heteroatoms. In some embodiments, branching may include a methyl (CH₃) group. In some embodiments, carbon chains may include an aryl group. In some embodiments, the unbranched or branched carbon chain may be substituted with a heteroatom. In some embodiments, the heteroatom may be oxygen. In some embodiments, the heteroatom may be a functional group, such as a carboxyl group or a hydroxyl group.

In some embodiments, the length of the acyl chain may be any value from about 12 carbon atoms to about 21 carbon atoms, or about 12, 13, 14, 15, 16, 17, 18, 19, 20, or 21 carbon atoms.

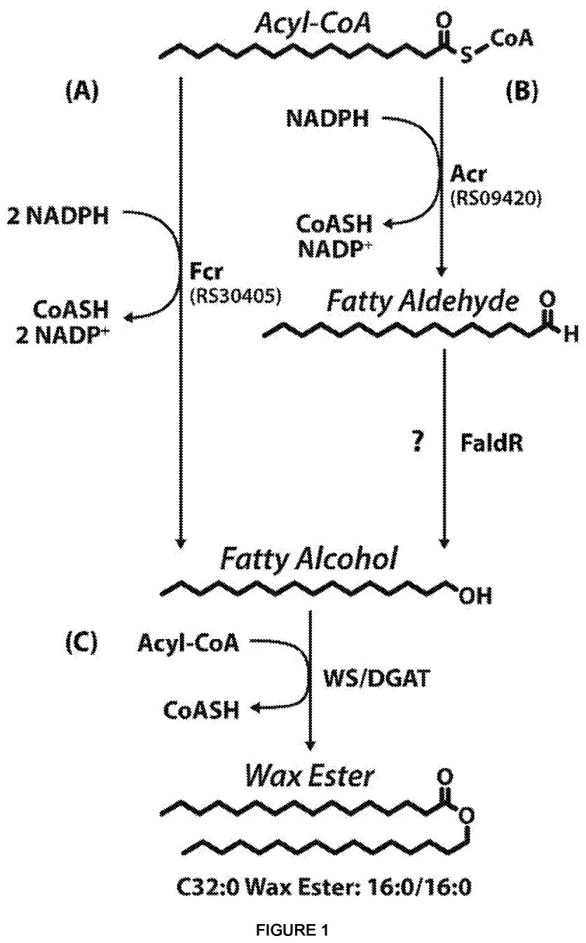

In some embodiments, the fatty alcohol may be generated through the action of a single enzyme (an “alcohol-forming fatty acyl-coenzyme A reductase”; FIG. 1A) or through consecutive reactions catalyzed by two enzymes (an “aldehyde-forming fatty acyl-CoA reductase” and a “fatty aldehyde reductase”; FIG. 1B).

In some embodiments, the WE may be generated through the esterification of a fatty alcohol and an acyl-CoA (FIG. 1C). A “fatty acyl-coenzyme A”, “acyl-coenzyme A”, or “acyl-CoA”, as used herein, refers to a group of coenzymes involved in fatty acid metabolism and having the general formula:

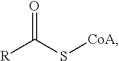

or a mixture of isomers thereof, where R may be a carbon chain, as described herein. In some embodiments, “acyl-CoA” may refer to a “CoA thioester” and may be formed by the esterification of a fatty acid with an acyl-CoA synthetase (CoASH) using adenosine triphosphate (ATP) and forming a thioester bond:

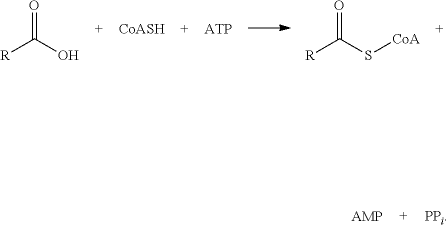

A “fatty acyl-coenzyme A reductase”, “alcohol-forming fatty acyl-CoA reductase,” “FAR”, “Fcr”, or “FcrA,” refers to an enzyme that catalyzes the synthesis of a fatty alcohol using acyl-coenzyme A and NADPH. In some embodiments, the Fcr may be obtained or derived from a Rhodococcus cell, such as a Rhodococcus jostii (for example, Rhodococcus jostii RHA1) or Rhodococcus opacus (for example, Rhodococcus opacus PD630). In some embodiments, the Rhodococcus jostii RHA1 may have a genomic sequence as set forth in NC_008268.1. In some embodiments, the Fcr may be RS30405 or EC 1.2.1.84. In some embodiments, the Fcr may be RS09420. In some embodiments, the Rhodococcus Fcr may have a sequence as set forth in Accession Nos. WP_011598195 or ABG97998. In some embodiments, the Rhodococcus FcrA may have a polynucleotide sequence as set forth in SEQ ID NO: 1, or a sequence substantially identical thereto. In some embodiments, the Rhodococcus Fcr may have a polypeptide sequence as set forth in SEQ ID NO: 2, or a sequence substantially identical thereto. In some embodiments, the Rhodococcus Fcr may have a nucleic acid sequence encoding an amino acid sequence as set forth in SEQ ID NO: 2, or a sequence substantially identical thereto.

In some embodiments, the Fcr may be obtained or derived from a Mycobacterium cell, such as a Mycobacterium tuberculosis. In some embodiments, the Mycobacterium Fcr may be Rv3391 or Rv1543 [59]. In some embodiments, the Mycobacterium Fcr may have a sequence as set forth in Accession Nos. AIR16184 or KBJ34903. In some embodiments, the Mycobacterium Fcr may have a polypeptide sequence as set forth in SEQ ID NO: 12 or 13, or a sequence substantially identical thereto. In some embodiments, the Mycobacterium Fcr may have a nucleic acid sequence encoding an amino acid sequence as set forth in SEQ ID NO: 12 or 13, or a sequence substantially identical thereto.

In some embodiments, the Fcr may be obtained or derived from a Marinobacter cell, such as a Marinobacter aquaeolei. In some embodiments, the Marinobacter Fcr may be Maqu_2507 [60] or Maqu_2220 [61]. In some embodiments, the Marinobacter Fcr may have a sequence as set forth in Accession Nos. YP_959769.1 or WP_011785687. In some embodiments, the Marinobacter Fcr may have a polypeptide sequence as set forth in SEQ ID NOs: 9 or 10, or a sequence substantially identical thereto. In some embodiments, the Marinobacter Fcr may have a nucleic acid sequence encoding an amino acid sequence as set forth in SEQ ID NOs: 9 or 10, or a sequence substantially identical thereto.

In some embodiments, the Fcr may be obtained or derived from an Acinetobacter cell, such as an Acinetobacter calcoaceticus. In some embodiments, the Acinetobacter Fcr may be Acr1 [62]. In some embodiments, the Acinetobacter Fcr may have a sequence as set forth in Accession No. WP_004923651, or a sequence substantially identical thereto. In some embodiments, the Acinetobacter Fcr may have a polypeptide sequence as set forth in SEQ ID NO: 11, or a sequence substantially identical thereto. In some embodiments, the Acinetobacter Fcr may have a nucleic acid sequence encoding an amino acid sequence as set forth in SEQ ID NO: 11, or a sequence substantially identical thereto.

In some embodiments, the Fcr may be recombinant. In some embodiments, the Fcr may be heterologous. In some embodiments, the Fcr may be a fragment having catalytic activity.

A “wax synthase” or “WS,” as used herein, refers to an enzyme that catalyzes the synthesis of WE from a fatty alcohol. In some embodiments, the WS may be obtained or derived from a Rhodococcus cell, such as a Rhodococcus jostii (for example, Rhodococcus jostii RHA1) or Rhodococcus opacus (for example, Rhodococcus opacus PD630). In some embodiments, the WS may be ws1, ws2, atfA, or atfA2.

In some embodiments, the ws1 may have a sequence as set forth in Accession No. ABO21020; the ws2 may have a sequence as set forth in Accession No. ABO21021; the atfA may have a sequence as set forth in Accession No. WP_004922247; or the atfA2 may have a sequence as set forth in Accession No. WP_011589085, or a sequence substantially identical thereto to any of the aforementioned sequences.

In some embodiments, the ws1 may have a nucleic acid sequence as set forth in SEQ ID NO: 4, or a sequence substantially identical thereto. In some embodiments, the ws1 may have an amino acid sequence as set forth in SEQ ID NO: 4, or a sequence substantially identical thereto. In some embodiments, the ws1 may have a nucleic acid sequence encoding an amino acid sequence as set forth in SEQ ID NO: 4, or a sequence substantially identical thereto.

In some embodiments, the ws2 may have a nucleic acid sequence as set forth in SEQ ID NO: 5, or a sequence substantially identical thereto. In some embodiments, the ws2 may have an amino acid sequence as set forth in SEQ ID NO: 6, or a sequence substantially identical thereto. In some embodiments, the ws2 may have a nucleic acid sequence encoding an amino acid sequence as set forth in SEQ ID NO: 6, or a sequence substantially identical thereto.

In some embodiments, the atfA may have an amino acid sequence as set forth in SEQ ID NO: 7, or a sequence substantially identical thereto. In some embodiments, the atfA may have a nucleic acid sequence encoding an amino acid sequence as set forth in SEQ ID NO: 7, or a sequence substantially identical thereto.

In some embodiments, the atfA2 may have an amino acid sequence as set forth in SEQ ID NO: 8, or a sequence substantially identical thereto. In some embodiments, the atfA2 may have a nucleic acid sequence encoding an amino acid sequence as set forth in SEQ ID NO: 8, or a sequence substantially identical thereto.

In some embodiments, the WS may be recombinant. In some embodiments, the WS may be heterologous. In some embodiments, the WS may be a fragment having catalytic activity.

The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule which is expressed using a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as ‘recombinant’ therefore indicates that the nucleic acid molecule has been manipulated using genetic engineering, i.e. by human intervention. Recombinant nucleic acid constructs may for example be introduced into a host cell by transformation. Such recombinant nucleic acid constructs may include sequences derived from the same host cell species or from different host cell species, which have been isolated and reintroduced into cells of the host species. Recombinant nucleic acid construct sequences may become integrated into a host cell genome, either because of the original transformation of the host cells, or as the result of subsequent recombination and/or repair events.

As used herein, “heterologous” in reference to a nucleic acid or protein is a molecule that has been manipulated by human intervention so that it is located in a place other than the place in which it is naturally found. For example, a nucleic acid sequence from one species may be introduced into the genome of another species, or a nucleic acid sequence from one genomic locus may be moved to another genomic or extrachromasomal locus in the same species. A heterologous protein includes, for example, a protein expressed from a heterologous coding sequence or a protein expressed from a recombinant gene in a cell that would not naturally express the protein.

The terms “nucleic acid” or “nucleic acid molecule” encompass both RNA (plus and minus strands) and DNA, including cDNA, genomic DNA, and synthetic (e.g., chemically synthesized) DNA. The nucleic acid may be double-stranded or single-stranded. Where single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. One example of a modified RNA included within this term is phosphorothioate RNA. By “DNA” is meant a sequence of two or more covalently bonded, naturally occurring or modified deoxyribonucleotides. By “cDNA” is meant complementary or copy DNA produced from an RNA template by the action of RNA-dependent DNA polymerase (reverse transcriptase). Thus a “cDNA clone” means a duplex DNA sequence complementary to an RNA molecule of interest, carried in a cloning vector. By “complementary” is meant that two nucleic acids, e.g., DNA or RNA, contain a sufficient number of nucleotides which are capable of forming Watson-Crick base pairs to produce a region of double-strandedness between the two nucleic acids. Thus, adenine in one strand of DNA or RNA pairs with thymine in an opposing complementary DNA strand or with uracil in an opposing complementary RNA strand. It will be understood that each nucleotide in a nucleic acid molecule need not form a matched Watson-Crick base pair with a nucleotide in an opposing complementary strand to form a duplex. A nucleic acid molecule is “complementary” to another nucleic acid molecule if it hybridizes, under conditions of high stringency, with the second nucleic acid molecule.

A “protein,” “peptide” or “polypeptide” is any chain of two or more amino acids, including naturally occurring or non-naturally occurring amino acids or amino acid analogues, regardless of post-translational modification (e.g., glycosylation or phosphorylation). An “amino acid sequence”, “polypeptide”, “peptide” or “protein” of the invention may include peptides or proteins that have abnormal linkages, cross links and end caps, non-peptidyl bonds or alternative modifying groups. Such modified peptides are also within the scope of the invention. The term “modifying group” is intended to include structures that are directly attached to the peptidic structure (e.g., by covalent coupling), as well as those that are indirectly attached to the peptidic structure (e.g., by a stable non-covalent association or by covalent coupling to additional amino acid residues, or mimetics, analogues or derivatives thereof, which may flank the core peptidic structure). For example, the modifying group can be coupled to the amino-terminus or carboxy-terminus of a peptidic structure, or to a peptidic or peptidomimetic region flanking the core domain. Alternatively, the modifying group can be coupled to a side chain of at least one amino acid residue of a peptidic structure, or to a peptidic or peptido-mimetic region flanking the core domain (e.g., through the epsilon amino group of a lysyl residue(s), through the carboxyl group of an aspartic acid residue(s) or a glutamic acid residue(s), through a hydroxy group of a tyrosyl residue(s), a serine residue(s) or a threonine residue(s) or other suitable reactive group on an amino acid side chain). Modifying groups covalently coupled to the peptidic structure can be attached by means and using methods well known in the art for linking chemical structures, including, for example, amide, alkylamino, carbamate or urea bonds.

A “substantially identical” sequence is an amino acid or nucleotide sequence that differs from a reference sequence only by one or more conservative substitutions, as discussed herein, or by one or more non-conservative substitutions, deletions, or insertions located at positions of the sequence that do not destroy the biological function of the amino acid or nucleic acid molecule. Such a sequence can be any value from 60% to 99%, or more generally at least 60%, 65%, 75%, 80%, 85%, 90%, or 95%, or as much as 96%, 97%, 98%, or 99% identical when optimally aligned at the amino acid or nucleotide level to the sequence used for comparison using, for example, the Align Program (Myers and Miller, CABIOS, 1989, 4:11-17) or FASTA. For polypeptides, the length of comparison sequences may be at least 2, 5, 10, or 15 amino acids, or at least 20, 25, or 30 amino acids. In alternate embodiments, the length of comparison sequences may be at least 35, 40, or 50 amino acids, or over 60, 80, or 100 amino acids. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and PrettyBox. Such software matches similar sequences by assigning degrees of homology to various substitutions, deletions, substitutions, and other modifications.

Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-CI, pH 7.6, lx Denhardt's solution, 10% dextran sulfate, and 0.1% SDS, at a temperature of 42° C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference. Substantially identical sequences may for example be sequences that are substantially identical to any one of SEQ ID NOs: 1 to 13, or to homologous sequences found in other organisms.

A fatty alcohol, WE, Fcr or WS may be produced in a suitable “host cell,” such as a bacterial, fungal, yeast, plant, algal, insect, amphibian, or animal cell. In some embodiments, the yeast cell may be, without limitation, Saccharomyces cerevisiae [56] or Yarrowia lipolitica. In some embodiments, the plant cell may be, without limitation, Arabidopsis thaliana [57] or tobacco [58]. In some embodiments, the bacterial cell may be a non-pathogenic cell. In some embodiments, the bacterial cell may be an avirulent pathogenic cell that is not capable of infecting a host. In some embodiments, the host cell may be a mycolic acid-containing bacterial cell. In some embodiments, the host cell may be, without limitation, a Streptomyces, Corynebacterium, Mycobacterium, Acinetobacter, or Marinobacter cell. In some embodiments, the host cell may be, without limitation, a Rhodococcus cell, such as a Rhodococcus jostii (for example, Rhodococcus jostii RHA1) or Rhodococcus opacus. In some embodiments, the Rhodococcus opacus may be a Rhodococcus opacus PD630, as deposited for example under DSM No.: 44193). In some embodiments, the host cell may be an Escherichia cell, such as an Escherichia coli. In some embodiments, the host cell may be a cell that naturally expresses a fatty acyl-coenzyme A reductase. In some embodiments, the host cell may be a cell that expresses a recombinant fatty acyl-coenzyme A reductase. In some embodiments, the host cell may be a cell that produces acyl-CoA. In some embodiments, the host cell that produces acyl-CoA may be a cell which produces a Fcr and/or WS, for example, a cell which produces the Fcr and/or WS naturally or in which the Fcr and/or WS has been recombinantly introduced.

By “conditions suitable for increased production of a fatty alcohol,” as used herein, is meant growth of a host cell in, for example, M9 minimal medium which may be supplemented with trace elements, thiamin, and any value from about 1 g/L to about 400 g/L, for example about 4 g/L, glucose as growth substrate as described herein or known in the art. In carbon-limited (C⁻) conditions, the M9 medium may contain additional amounts of a nitrogen source, such as any value from about 1 g/L to about 50 g/L, for example about 1 g/L ammonium chloride, ammonium sulfate, ammonium hydroxide, urea, ammonium nitrate, whey or other suitable nitrogen source. In nitrogen-limiting (N⁻) conditions, the M9 medium may contain reduced amounts of a nitrogen source, such as any value from about 0.01 g/L to about 1 g/L, for example about 0.05 g/L ammonium chloride, ammonium sulfate, ammonium hydroxide, urea, ammonium nitrate, whey or other suitable nitrogen source. In some embodiments, carbon sources other than glucose may include, without limitation, various sugars, aromatics, lignocellulosic biomass or conversion products thereof, fatty acids, TAGs, hydrocarbons, and industrial waste streams, such as pulp, plant biomass waste, manure, food oils, etc. In some embodiments, suitable conditions may include C⁻ conditions. In some embodiments, suitable conditions may include growing a host cell expressing a recombinant Fcr and a recombination WS under C⁻ conditions in exponential phase. In some embodiments, suitable conditions may include N⁻ conditions.

In some embodiments, by “increased production” is meant an increase in the production or “overproduction” of a fatty alcohol or a WE in a host cell, when grown in C⁻ conditions, when compared to a cell grown in non-C⁻ conditions, such as N⁻ conditions. In some embodiments, by “increased production” or “overproduction” is meant an increase in the production of a fatty alcohol or a WE in a host cell expressing or overexpressing a recombinant Fcr compared to a cell not expressing a recombinant Fcr. In some embodiments, by “increased production” or “overproduction” is meant an increase in the production of a fatty alcohol or a WE in a host cell expressing or overexpressing a recombinant Fcr in combination with a recombinant WS, compared to a cell not expressing a recombinant Fcr or a recombinant WS. The recombinant Fcr or WS may be expressed at a level greater than that expressed naturally by a Fcr or WS in the host cell, for example, the level of expression of a recombinant Fcr or a recombinant WS may be any value between about 5% and about, 100%, or any value therebetween, such as 5%, 10%, 15%, 20%, 25%, 30%, 35%, 40%, 45%, 50%, 55%, 60%, 65%, 70%, 75%, 80%, 85%, 90%, 95%, 100% or may be over 100%, greater than that produced naturally by a host cell that does not express a recombinant Fcr or a recombinant WS. In some embodiments, by “increased production” or “overproduction” is meant an increase in the production of a WE in a host cell, as measured by cell dry weight (CDW). In some embodiments, a host cell that exhibits increased production of a WE, may exhibit over 10% CDW of WEs, such as over 12%, 13%, 14%, 15%, 20%, 25% or more.

Other growth conditions may depend on, or be specific to, the host cell, for example, some Rhodococcus strains may be grown at 30° C. while shaking at 200 rpm. In some embodiments, the growth temperature may be any value from about 10° C. to about 30° C. In some embodiments, the pH may be any value from about 5 to about 8. In some embodiments, the dissolved oxygen may be any value from about 20% to about 100%. In some embodiments, the host cell may be grown in a shake flask or in a bioreactor that is, for example, run in a batch or fed-batch manner. It is to be understood that, in general, conditions suitable for increased production of a fatty alcohol are also suitable for production of a WE.

Fatty alcohols and wax esters may be isolated from a host cell by any suitable techniques, as described herein or known in the art. In some embodiments, fatty alcohols and wax esters may be isolated from a host cell using techniques suitable for large scale or commercial scale isolation. For example, organic solvent extraction techniques (such as those using choloroform, hexanes, etc.), supercritical CO₂ techniques, and/or mechanical techniques may be used.

A “vector” is a DNA molecule derived, for example, from a plasmid, bacteriophage, or mammalian or insect virus, or artificial chromosome, into which a nucleic acid molecule, for example, a Fcr or WS, may be inserted. A vector may contain one or more unique restriction sites and may be capable of autonomous replication in a defined host or vehicle organism such that the cloned sequence is reproducible. A vector may be a DNA expression vector, i.e, any autonomous element capable of directing the synthesis of a recombinant polypeptide, and thus may be used to express a polypeptide, for example a Fcr or WS polypeptide, in a host cell. DNA expression vectors include bacterial plasmids and phages and mammalian and insect plasmids and viruses. A vector may be capable of integrating into the genome of the host cell (an “integrative vector”), such that any modification introduced into the genome of the host cell by the vector becomes part of the genome of the host cell. A vector may be incapable of integrating into the genome of the host cell, and therefore remain as an autonomously replicating unit, such as a plasmid. Accordingly, a vector may be an “expression vector” or “cloning vector” or “integrative vector” designed for gene expression or replication in cells and may include without limitation plasmids, viral vectors, cosmids, bacterial vectors, or artificial chromosomes. Integrative vectors may include without limitation pSET152. Expression vectors, may include without limitation a plasmid expression vector such as the pTipQC2 vector. In some embodiments, the Fcr and the WS may both be integrated into the genome of the host cell. In alternative embodiments, the Fcr and the WS may both be on a single or on separate plasmids. In alternative embodiments, the one of the Fcr and the WS may both be on a plasmid and the other may both be integrated into the genome of the host cell.

A vector may be operably linked to a promoter. By “operably linked” is meant that a gene and a regulatory sequence(s) are connected in such a way as to permit gene expression when the appropriate molecules (e.g., transcriptional activator proteins) are bound to the regulatory sequence(s).

By “promoter” is meant the minimal sequence sufficient to direct transcription. Promoters usually lie 5′ from the sequence to be read and regulate the transcription rate of a gene. Accordingly, a promoter may include a binding site in a DNA chain at which RNA polymerase binds to initiate transcription of messenger RNA by one or more nearby structural genes. A promoter may include without limitation constitutive (for example, Pₙᵢₜ) or inducible (for example, P_{tipA}) promoters. An inducible promoter refers to a promoter where initiation of transcription can be induced by for example addition of a compound to the growth medium or by changing the temperature. Also included are those promoter elements which are sufficient to render promoter-dependent gene expression controllable for cell-type specific, tissue-specific or inducible by external signals or agents; such elements may be located in the 5′ or 3′ regions of the native gene. In some embodiments, the promoter may include without limitation P_{sod}, P_{ermEp1} P_{ermE2}, P_{ermE1*}, P_{rpsL}, P_{SF14P}, P_{nitA7}, Pₙᵢₜ, P_{tipA}, P_{RS04990}, P_{RS05290}, Pᵣₒ₀₁₁₇₈, P_{RS19805}, P_{RS19555}, P_{RS19805}, P_{RS20995}, P_{RS21170}, P_{RS30940}, P_{RS36130}, P_{RS44810}, P_{RS11020}, P_{RS30980}, P_{RS05505}, P_{RS21725}, P_{RS30245}, P_{RS10475}, P_{RS09620}, P_{RS09350}, or P_{RS1947} In some embodiments, the Pₙᵢₜ promoter may be used with the primers 5′-GGTCGACTCTAGAGCGGCCGCTCACTCTTCTGCTCGGCC-3′ (SEQ ID NO: 14) and 5′-AACAAAATTATTTCTAGACGATATCGCCGTCCATTATACCTCCTCACGTGACGTGAG-3′ (SEQ ID NO: 15).

In some embodiments, the integrative vector pSET152 may be used in combination with the constitutive promoter Pₙᵢₜ. In some embodiments, the expression vector pTipQC2 may be used in combination with the inducible promoter P_{tipA}.

Methods for expressing a polypeptide in a host cell are known in the art. A polypeptide may be heterologously expressed from a polynucleotide sequence cloned into the genome of said host cell or it may be transformed or transfected into said host cell. Expression may occur in a transient manner. When the polynucleotide encoding the polypeptides may be cloned into the genome, an inducible promoter may be cloned as well to control expression of the polypeptide. The term “overexpression” may refer to “an amount of deliberate protein production greater than that seen in the “wild-type cell”, and the term “wild-type” may refer to the phenotype of the typical form of a species as it occurs in nature. Overexpression may lead to increased production of a fatty alcohol or WE as described herein.

The polypeptides, nucleic acid molecules, vectors and/or host cells may be provided in a kit, together with instructions for use. For example, the nucleic acid molecules may be provided with instructions for construction of a vector; the vectors may be provided with instructions for generation of a host cell that can express or overexpress a Fcr or WS; and/or the host cells may be provided with instructions for growth in suitable conditions for the increased expression of a fatty alcohol or WE.

Fatty alcohols or wax esters may be useful in personal care products, cosmetics, coatings, foods, lubricants, pharmaceutical formulations, metal processing, or candle wax. The term “personal care product” is used herein as it is normally understood to a person of ordinary skill in the art and often refers to “toiletries” or “consumer products uses in personal hygiene and for beautification”.

The present invention will be further illustrated in the following examples.

Materials and Methods

Strains and Culture Conditions—

Escherichia coli DH5a was used to propagate DNA. E. coli S17.1 was used to conjugate pK18-derived plasmids into RHA1. E. coli strains were grown in LB broth at 37° C., 200 rpm. RHA1 was grown at 30° C. while shaking at 200 rpm. For protein production, RHA1 was grown in LB. For lipid production, RHA1 was grown in M9 minimal medium supplemented with trace elements, thiamin, and 4 g/L glucose as growth substrate [39, 40]. In carbon-limited (C⁻) conditions, the M9 medium contained 1 g/L ammonium chloride. In nitrogen-limiting (N⁻) conditions, this concentration was 0.05 g/L, as previously described [3]. For solid medium, LB broth was supplemented with Bacto agar (1.5% [w/v]; Difco). Media were further supplemented with 100 pg/mL ampicillin (E. coli carrying pTip-derived plasmids), 50 pg/mL kanamycin (E. coli carrying pK18-derived plasmids), 34 pg/mL chloramphenicol (RHA1 carrying pTip-derived plasmids) or 10 pg/mL neomycin (RHA1 carrying pK18-derived plasmids) as appropriate.

Reagents—

Enzymes for cloning were purchased from New England Biolabs unless otherwise noted. Primers were ordered from Integrated DNA Technologies. Chemicals were of at least reagent grade unless otherwise noted. Buffers were prepared using water purified on a Barnstead NANOpure UV apparatus to a resistivity of greater than 17 MΩ cm.

DNA Manipulation, Plasmid Construction, and Gene Deletion—

DNA was isolated, manipulated, and analyzed using standard protocols [40]. E. coli and RHA1 were transformed with DNA by electroporation using a MicroPulser with GenePulser cuvettes (Bio-Rad). To produce a C-terminal His₆-tag FcrA, RHA1_RS30405 was amplified from RHA1 genomic DNA using Phusion Polymerase™ with the primers 5′-TTTAAGAAGGAGATATACATATGGCCACCTACCTCGTCACCG-3′ (SEQ ID NO: 16) and 5′-ATGGTGATGGTGATGCTCGAGGCTGCTGCCCTGGAAATACAAGTTTTCTCTGCCGCTGCTC CA-GTGGGTGCCGGGCAC-3′ (SEQ ID NO: 17). The resulting amplicon was inserted into pTip-QC2 linearized with Nde1 and Xho1 using Gibson Assembly. Tagless FcrA was produced as above using the primers 5′-TTTAAGAAGGAGATATACATATGGCCACCTACCTCGTCACC-3′ (SEQ ID NO: 18) and 5′-GTGCCGGTGGGTCGACTA-GTCACCAGTGGGTGCCGGG-3′ (SEQ ID NO: 19). The nucleotide sequence of the cloned genes was verified. The ΔfcrA mutant was constructed using a sacB counter selection system [41]. Two 500 bp flanking regions of RHA1_RS30405 were amplified from RHA1 genomic DNA using the upstream primers 5′-TGAGGTGCGAGGACGTGGATCTCGGCTG-3′ (SEQ ID NO: 20) and 5′-CGGGTACCGAGCTCGAATTCGTCTCCGCGCC-ATCACGC-3′ (SEQ ID NO: 21) and the downstream primers 5′-GCTATGACATGATTACGAATTCGGCCGGGGTGCGG-GTCA-3′ (SEQ ID NO: 22) and 5′-ACGTCCTCGCACCTCAGCACACACCGGAACCC-3′ (SEQ ID NO: 23). The resulting amplicons were inserted into pK18mobsacB linearized with EcoR1 using Gibson Assembly. The nucleotide sequence of the resulting construct was verified. Kanamycin-sensitive/sucrose-resistant colonies were screened using PCR and the gene deletion was confirmed by sequencing.

Construction of Modular, Integrative Vector Producing mCherry—

A modular, integrative vector producing mCherry was constructed to simplify the cloning of genes such as fcrA into the vector. The mCherry gene was amplified from pMS689mCherry using the primers 5′-CTTTAAGAAGGAGATATACATATGGTGAGCAAGGGCGAG-3′ (SEQ ID NO: 24) and 5′-CACGGGTGCCGGT-GGGTCGACTAGTCACTTGTACAGCTCGTCCATG-3′ (SEQ ID NO: 25). The amplicon was inserted into Nde1/Spe1-linearized pTipQC2 using Gibson Assembly to yield pTip-mCherry. A modular expression cassette, including the RBS, reporter, and terminator, was then amplified from pTip-mCherry using the primers 5′GGCTGCAGGTCGACTCTAGAGCGGCCGCATCGATCATTGATATCGTCTAGAAATAATTTT G-TTTAACTTTAAG-3′ (SEQ ID NO: 26) and 5′-GCGTTGGCCGATTCATTAATACTAGAGTCCCGCTGAGG-3′ (SEQ ID NO: 27). The amplicon was inserted into Not1/Ase1-linearized pSET-152 using Gibson Assembly to yield pSYN000-mCherry. The PNit promoter was then amplified from pTipQC2 using the primers 5′-GGTCGACTCTAGAGCGGCCGC-TCACTCTTCTGCTCGGCC-3′ (SEQ ID NO: 28) and 5′-AACAAAATTATTTCTAGACGATATCGCCGTCCATTATACCTCCTC-ACGTGACGTGAG-3′ (SEQ ID NO: 29). The amplicon was inserted into Not1/EcoRV-linearized pSYN000-mCherry using Gibson Assembly, resulting in pSYN001-mCherry. To clone RS304005 (fcrA) (SEQ ID NO. 1) into this vector, fcrA was amplified from RHA1 genomic DNA using the primers 5′-TTTAAGAAGGAGATATACATATGGCCACCTACCTCGTCACC-3′ (SEQ ID NO: 30) and 5′-GTGCCGGTGGGTCGACTAGTCACCAGTGGGTGCCGGG-3′ (SEQ ID NO: 31). The amplicon was inserted into Nde1/Spe1-linearized pSYN001-mCherry using Gibson Assembly, resulting in pSYN001-fcrA. The nucleotide sequence of all constructs was verified.

FcrA Production and Purification—

RHA1 freshly transformed with pTip-fcrA were grown overnight in LB. These cultures were used to inoculate 1 L fresh LB medium to an optical density at 600 nm (OD₆₀₀) of 0.05. Cultures were grown to an OD₆₀₀˜0.8, the expression of fcrA was induced with 10 pg/mL of thiostrepton, and cultures were incubated for a further 24 h. Cells were harvested by centrifugation and stored at −80° C.

Cells from 2 L of culture were suspended in 40 mL of lysis buffer (50 mM Na-phosphate, pH 8.5, 500 mM NaCl, 2.5 mM imidazole) containing complete Mini EDTA-Free protease inhibitors (Roche Diagnostics). The cell suspension was split between four 15 mL conical tubes each containing˜1 mL 0.1 mm zirconium/silica beads and −1 mL 0.5 mm glass beads (BioSpec Products) and subjected to three rounds of bead-beating at 5-6 m/s using a FastPrep®-24 (MP Biomedicals) with 5 min on ice between rounds. The lysate was centrifuged (4000×g for 5 min) to remove unbroken cells. The supernatant lysate was removed and stored on ice. The pellets were suspended in 5 mL lysis buffer and subjected to another 3 rounds of bead-beating. The supernatants were combined and further clarified by ultracentrifugation (40,000×g for 60 min) and passage through a 0.22 μm filter.

The clarified lysate was incubated with 5 mL Ni Sepharose 6 fast flow resin (GE Healthcare Bio-Sciences) for 3 h. After incubation, the resin was poured into a column, then washed with 50 mL lysis buffer, 50 mL lysis buffer containing 50 mM imidazole, and 15 mL lysis buffer containing 75 mM imidazole. FcrA was eluted with 15 mL lysis buffer containing 250 mM imidazole. FcrA-containing fractions, as judged by SDS-PAGE, were pooled, exchanged into 50 mM Na-phosphate, pH 8.5, 500 mM NaCl, and concentrated to −10 mg/mL using an Amicon Ultra-15 centrifugal filtration unit (Merck KGaA) equipped with a 30 kDa cut-off membrane. Protein was flash frozen as beads in liquid nitrogen and stored at −80° C. Protein concentration was determined by Micro BCA (Thermo Fisher Scientific, Rockford, Ill.) and by absorbance at 280 nm.

Mass Spectrometry—

Purified FcrA (˜4 pg/mL in 5% acetonitrile, 0.1% formic Acid (v/v)) was injected onto a 5 mm C4 column connected to a Waters Xevo GS-2 QTof mass spectromter via a NanoAquity UPLC system operated at 20 μL/min. Samples were eluted in a 40 μL gradient of 5-100% acetonitrile at 20 μL/min. MS spectra were summed and deconvoluted using Waters' MaxEnt algorithm.

FcrA Activity Assays—

FcrA activity was evaluated using two spectrophotometric assays [42]. Assays were performed at 25° C. in 1 mL 20 mM MOPS, 80 mM NaCl, pH 7.0 (I=0.1 M) containing 5 μM acyl-CoA and 200 μM NADPH. Reactions were initiated by the addition of FcrA to a final concentration of 0.5 to 2 μM. In one assay, the oxidation of NADPH was followed at 340 nm (E=6.3 mM mM⁻¹ cm⁻¹ [43]). In the second assay, the reaction mixture also contained 0.1 mg/mL DTNB, and the formation of NTB²⁻ was followed at 412 nm (E=14.15 mM⁻¹ cm⁻¹ for NTB²⁻[44]). Reaction rates were calculated from progress curves using Cary WinUV Kinetics Application (Agilent). The pH-dependence of the reaction was evaluated using the DTNB assay and a series of 20 mM Good's buffers (MES, MOPS, HEPPS, or TAPS), 80 mM NaCl (I=0.1 M), pH 6 to 9.

Production of Neutral Lipids and WEs—

Fresh media were inoculated to an OD₆₀₀ of 0.1 using washed RHA1 cells harvested from cultures grown overnight in C⁻ conditions. For analyses of C⁻ cultures, cells were harvested during exponential growth, approximately 24 h after inoculation. For analyses of N⁻ cultures, cells were harvested after approximately 72 h [3]. To induce nitric oxide (NO) stress, cells were grown under C⁻ conditions to an OD₆₀₀ of 0.5-0.6 at which point sodium nitroprusside (SNP) was added to the cultures to a concentration 1 mM at two-hour intervals for six hours. NO-stressed cells were harvested two hours after the final addition of SNP. For RHA1 transformed with pTip-fcrA or empty pTipQC2, cultures were grown as above, except that when they reached an OD₆₀₀ between 0.6-1.0, thiostrepton was added to 10 μg/mL. Cells were pelleted at 4,000×g for 30 min at 4° C., washed with distilled water, and stored at −80° C.

Lipid Extraction and Thin Layer Chromatography (TLC)—

Frozen cell pellets were lyophilized for 24 to 48 h using a FreeZone 2.5 (LABCONCO). Dried cells were suspended in water and sonicated to lyse cells. Myristyl myristate (Nu-Chek Prep) and/or ethyl myristate (Sigma) was added as a standard. Total lipids were extracted from lysate using 2:1 chloroform:methanol with 1% acetic acid [45]. The organic phase was collected, and dried using a rotary evaporator (Buchi) and/or nitrogen gas. Dried extracts were suspended in chloroform and stored at −20° C. Lipid extracts were analyzed using silica-TLC and a mobile phase of 90:6:1 v/v of hexane/diethyl ether/acetic acid. Lipids were visualized by staining with 10% cupric sulphate in 8% aqueous phosphoric acid (v/v) and charring for 5 min at 200° C.

Isolation, Fractionation, and Gravimetric Quantification of Neutral Lipids—

Neutral cellular lipids were purified using flash chromatography. Total lipid extracts were applied to a column of silica resin (10 mg lipids per 300 mg resin [46]) equilibrated with 5 column volumes (CV) of chloroform. Neutral lipids eluted with 5 CV of chloroform, were dried as described above, and quantified by weight using an AT200 analytical balance (METTLER). The neutral lipids were suspended in hexanes and fractionated using silica equilibrated with 5 CV of hexanes. Neutral lipid extracts were applied to the column and the hydrocarbon fraction was eluted with 10 CV of hexanes, the WE fraction with 10 CV of 98:2 v/v of hexanes/diethyl ether, and the remaining TAGs and neutral lipids with 10 CV of chloroform. The fractions were dried, weighed, and suspended in chloroform for storage at −20° C.

To analyze fatty alcohols produced in vitro, quenched reactions of 2 mL were extracted with 5 mL of 2:1 chloroform:methanol (v/v). The organic phase was collected, washed once with 1:1 H₂O:methanol, and three times with H₂O. The organic phase was removed and dried under nitrogen stream. The extract was suspended in pyridine and derivatized with tetramethylsilane for 1 h at 60° C. Derivatized extracts were analyzed by gas chromatography-mass spectrometry (GC/MS) as described below.

GC/Ms Analyses—

Analyses were performed using an Agilent 6890n gas chromatograph system fitted with an HP-5 MS 30 m×0.25 mm capillary column (Hewlett-Packard) and an Agilent 5973n mass-selective detector. The GC was operated at an injector temperature of 300° C., a transfer line temperature of 320° C., a quad temperature of 150° C., a source temperature of 230° C., and a helium flow rate of 1 mL/min. Samples of 1 μL were injected in splitless mode. For fatty alcohol analyses, the temperature program of the oven was 40° C. for 2 min, increased to 160° C. at a rate of 40° C. per min, then increased to 240° C. at a rate of 5° C. per min, and finally increased to 300° C. at a rate of 60° C. per min and held for 5 min. The mass spectrometer was operated in electron emission scanning mode at 40 to 800 m/z and 1.97 scans per second. For WE analysis, the temperature program of the oven was 40° C. for 2 min, increased to 180° C. at a rate of 40° C. per min, and then increased to 320° C. at a rate of 2.5° C. per min and held for 20 min. Derivatized fatty alcohols and WEs were identified using Chemstation E.02.02.1431 (Agilent) and the NIST08 Library. The identity of fatty alcohols was further verified using similarly derivatized authentic fatty alcohols. WEs were further identified by comparison to an authentic stearyl stearate standard and were quantified using a standard curve generated using palmityl myristate (Nu-Chek Prep).

Wax Ester Accumulation in Rhodococcus opacus PD630—

Lipid analysis and manipulation of PD630 was performed as described for RHA1.

Expression of fcrA Using Various Strength Constitutive Promoters—

P_{M6} was amplified using the primers 5′-GGTCGACTCTAGAGCGGCCGCACCGCTCTGGTCAGCGAC-3′ (SEQ ID NO: 32) and 5′-AACAAAATTATTTCTAG-ACGATATCTGGAGTCGGTTGACCAG-3′ (SEQ ID NO: 33). P_{T1} was amplified using the primers 5′-GGTCGACTCTAGAGC-GGCCGCACCGCTCTGGTCAGCGAC-3′ (SEQ ID NO: 34) and 5′-AACAAAATTATTTCTAGACGATATCCTTGCGACGAAAGGA-ACTC-3′ (SEQ ID NO: 35). P_{T2} was amplified using the primers 5′-GGTCGACTCTAGAGCGGCCGCACCGCTCTGGTCAGC-GAC-3′ (SEQ ID NO: 36) and 5′-AACAAAATTATTTCTAGACGATATCGAAAGGAACTCTACAACAGCGAC-3′ (SEQ ID NO: 37). M6, T1, and T2 promoters were Gibson cloned into Not1/EcoRV-linearized pSYN001-fcrA linearized to yield, pSYN002-fcrA, pSYN003-fcrA, pSYN004-fcrA, respectively. pSYN vectors were transformed into RHA1, and WE content was analysed in N− cultures using GC/MS as previously described.

Co-Expression of fcrA and Wax Synthases—

RHA1 was co-transformed with pSYN004-fcrA and pTip vectors carrying WSs using standard procedures. Transformed cells were grown in both C− and N− media. WS production was induced at an OD₆₀₀ between 0.3-0.5 as described, and cells were harvested in exponential and stationary for C− and N− cultures, respectively. WE content was analysed by GC/MS.

### Example 1: Production of WEs in RHA1

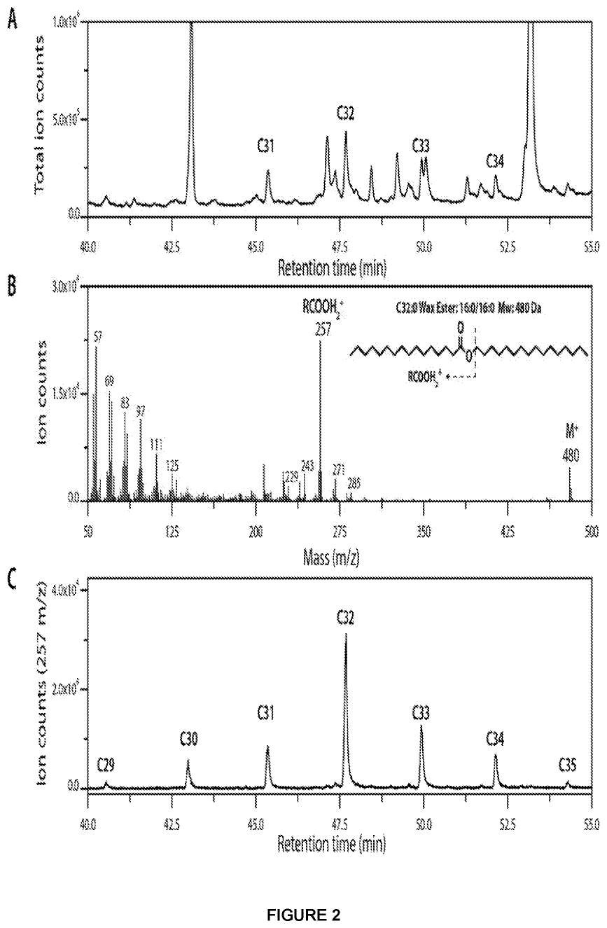

RHA1 was grown on glucose minimal medium (C⁻ conditions). Neutral lipids were extracted from exponentially growing cells and fractionated using flash chromatography. Initial GC/MS analyses revealed that cells contained saturated WEs ranging from 31-34 carbons in length, with C32 species being the most abundant (FIG. 2A). Using electron ionization mass spectrometry, WEs were analyzed using the RCOOH₂⁺ and RCO—H⁺. ions of their saturated and unsaturated fatty acid components, respectively [47]. For example, the length of the fatty acyl component of the C32 WEs varied from 14 to 18 carbons, as seen from the RCOOH₂⁺ ions with m/z values of 229, 243, 257, 271, 285, respectively (FIG. 2B). These ions indicated that ˜70% of the C32 WEs were C16:C16 species, while the remainder were a mixture of species varying from C14:C18 to C18:C14. Finally, using the highly abundant C16 ion, which was more abundant than the corresponding WE molecular ion, additional WEs were identified with 29, 30, and 35 carbon atoms that were not apparent in the total ion trace (FIG. 2C).

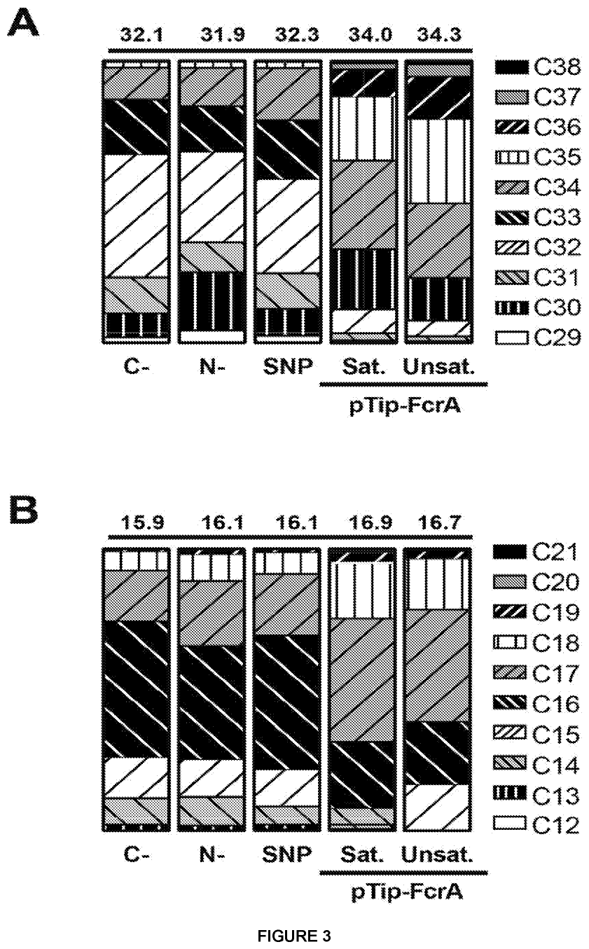

In summary, the most abundant WEs under C⁻ conditions contained 32 carbons (FIG. 3A). Moreover, C16 was the most abundant WE fatty acyl moiety in the detected WEs (FIG. 3B). No unsaturated fatty acids were detected under these conditions. Quantification by GC/MS revealed that the cells contained 2.2±0.2 μg of WEs/g cell dry weight (CDW). As shown in Table 1, the cells contained lower amounts of WEs (0.6±0.2 μg/g CDW) under N⁻ conditions, conditions under which they accumulate TAGs to over 50% of their CDW [1]. WEs produced under N⁻ conditions were similar in length and composition to those under C⁻ conditions (FIGS. 3A-B).

TABLE 1
WE levels^{a} in RHA1 strains under different growth conditions.

| | Growth condition | | |
|---|---|---|---|
| | N⁻ | C⁻ | SNP-treated |
| RHA1 strain | (stationary) | (exponential) | (exponential) |
| WT | 0.6 ± 0.2 | 2.2 ± 0.3 | 3.0 ± 0.3 |
| ΔfcrA | 0.7 ± 0.1 | 3.2 ± 0.5 | 0.5 ± 0.1 |

^{a}Experimental values represent μg WE/g CDW.

### Example 2: Bioinformatic Identification of FcrA

Having identified WEs in RHA1, the strain's genome was searched for homologs of fcr1 or fcr2 of M. tuberculosis, which encode fatty acyl-CoA reductases (FARs) [35]. A BLAST analysis identified RHA1_RS30405 and RHA1_RS09420 as reciprocal best hits of Fcr1 and Fcr2, respectively. RS30405 encodes a protein of 664 amino acid residues, identified here as FcrA, that shares 56% and 43% amino acid sequence identities with Fcr1 and Maqu_2507 from M. aquaeolei VT8, respectively. The three enzymes share a similar two-domain structure in which each domain contains a predicted nucleotide-binding site. RS30405 is conserved in all rhodococci whose genomes have been sequenced. RS09420 encodes a protein of 280 amino acids that shares 48% and 46% amino acid sequence identities with Fcr2 and Acr1 from A. calcoaceticus, respectively, as well as 46% identity with the second nucleotide-binding domain of FcrA.

Further studies of the transcript levels revealed that under N⁻ conditions, the transcript levels of both fcrA and RS09420 were low, with reads per kilobase per million mapped reads (RPKM) values between 6 and 11 in exponential and stationary phase. Moreover, fcrA appeared to be more highly expressed under C⁻ conditions, with RPKMs of 19±9 and 87±6 in exponential and stationary phase, respectively. By contrast, RS09420 transcript levels were also low under C⁻ conditions: the RPKM value was 9±5 in exponential phase and no transcripts were detected in stationary phase.

The RNA-seq data also provided insight into the operon structures of fcrA. Thus, RS30410, located immediately upstream of fcrA and encoding a putative membrane protein, was not co-transcribed with fcrA. Interestingly, RS30410 is conserved upstream of fcrA across the diverse phylogenetic clades of rhodococci [48], while the surrounding genomic context is not. By contrast, a homolog of RS30410 is not present upstream of fcr1 in mycobacterial species, suggesting that RS30410 is not essential for the activity of fcrA.

### Example 3: Production, Purification, and Characterization of Recombinant FcrA

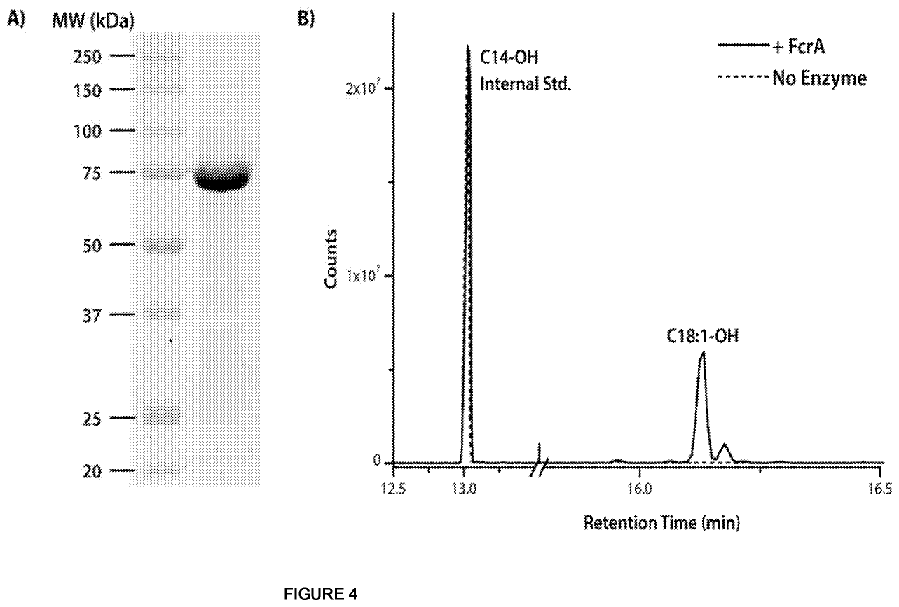

Because related two-domain enzymes completely reduce acyl-CoAs to fatty alcohols [42] and have been used in biotechnology applications [49, 50], further efforts were focused on exploring the function of FcrA, To investigate the activity of FcrA, it was first produced as a C-terminally His-tagged protein in RHA1 and purified to greater than 95% apparent homogeneity (FIG. 4A). The molecular mass of FcrA-His₆ was 73627 Da, which corresponds to the mass of the protein (73758 Da) less the N-terminal methionine (131 Da). Then, to evaluate the ability of FcrA to catalyze the formation of fatty alcohols, 1.4 μM FcrA was incubated with 400 μM NADPH and 100 μM oleoyl-CoA in 1 mL 20 mM Tris-HCl, pH 7.0, 50 mM NaCl for 20 h at room temperature. The reaction was quenched with 1 mL saturated NaCl and 100 μM of tetradecanol was added as a standard. Analysis of the extracted and derivatized reaction products by GC/MS revealed that FcrA converted oleoyl-CoA to oleyl alcohol (FIG. 4B). No alcohol was detected in control reactions in which FcrA was omitted. Further investigation, using a spectrophotometric assay following either the production of NTB²⁻ or the consumption of NADPH, showed that FcrA catalyzed the reduction of various acyl-CoAs. As with previously characterized FARs [35, 42], activity was maximal at pH 7.0. Moreover, no activity was detected when NADPH was substituted with NADH. Similar to Maqu_2507 [42], the rate of NADPH consumption was twice that of NTB²⁻ production, indicating that FcrA oxidizes two equivalents of NADPH for every molecule of CoA released. Among the tested substrates, FcrA had highest specific activity for stearoyl-CoA (C18-CoA), reducing it at a rate of 45±3 nmol/mg·min. The specific activity of FcrA dropped off with decreasing chain length of the acyl-CoA substrate (Table 2). By contrast, oleoyl-CoA (C18:1-CoA), a monounsaturated acyl-CoA, was reduced at a similar rate to stearoyl-CoA.

TABLE 2
Specific activities of FcrA with various acyl-CoA substrates.

| | | Specific Activity |
|---|---|---|
| Substrate | Chain Length | (nmol/min/(mg protein)) |
| Stearoyl-CoA | C18 | 45 ± 3 |
| Oleoyl-CoA | C18:1 | 41 ± 1 |
| Palmitoyl-CoA | C16 | 7.0 ± 0.1 |
| Lauroyl-CoA | C12 | 1.5 ± 0.3 |

### Example 4: Wax Ester Production in a ΔfcrA Mutant

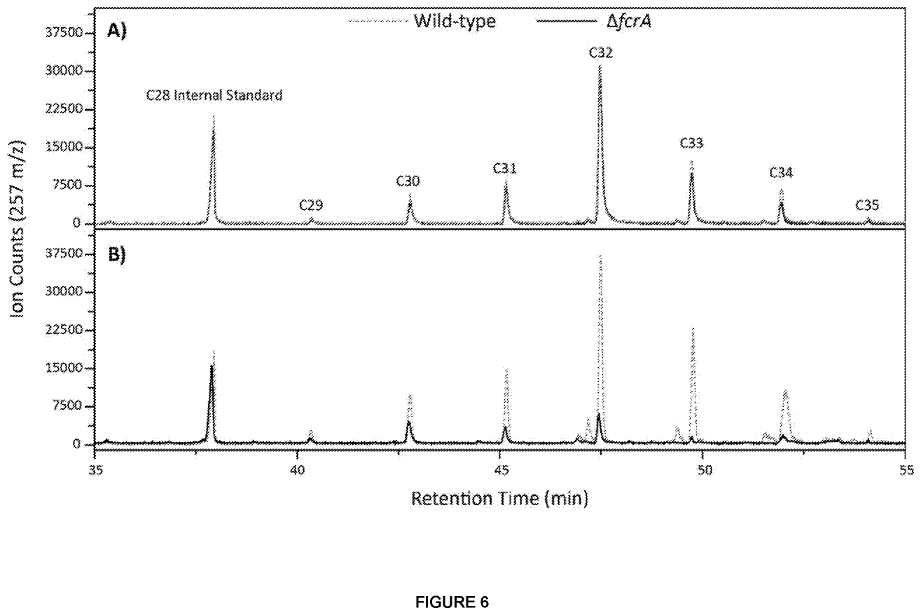

By deleting the fcrA gene in RHA1, the ΔfcrA mutant was created, which contained similar amounts of WEs as wild type (WT) RHA1 under both C⁻ and N⁻ conditions (Table 1). However, in M. tuberculosis, a Δfcr1 mutant did not show a decrease in WEs when starved for both carbon and nitrogen but did so under conditions of nitric oxide (NO) stress [35]. Therefore, the ability of the ΔfcrA mutant to produce WEs was tested in the presence of reactive nitrogen species in RHA1 by using SNP, to generate NO. When stressed with NO, WT cells growing exponentially on glucose minimal medium produced similar levels of WEs as non-stressed cells. The majority of the WE species observed in WT RHA1 are similar in chain length and composition to those seen under other growth conditions (FIGS. 3A-B). However, trace amounts of unsaturated C32-34 WEs were detected in the SNP-treated cells. The production of WEs in response to NO stress was strikingly different in the ΔfcrA mutant (FIGS. 6A-B), with a 6-fold reduction in accumulated WEs (Table 1).

### Example 5: Overexpression of fcrA with a Plasmid

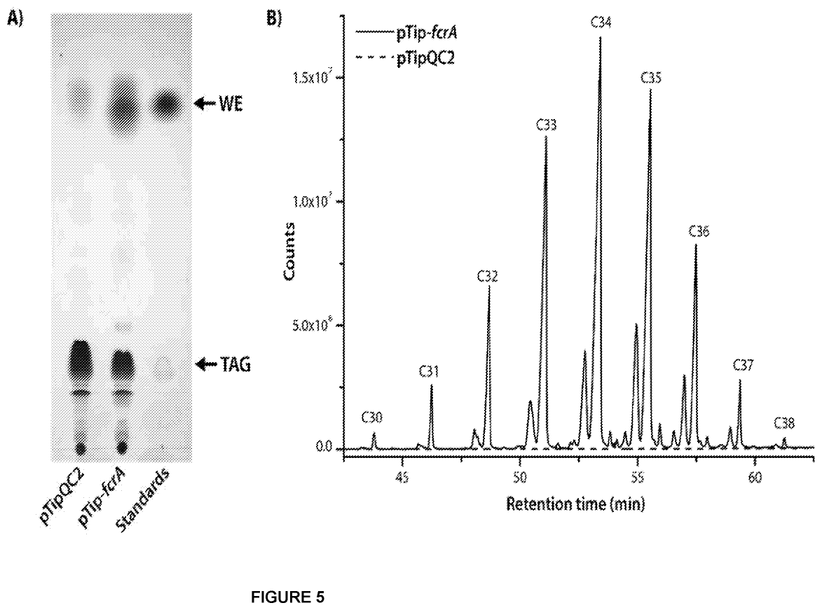

A tag-less form of FcrA was overproduced using a pTip vector. The experiment was performed under N⁻ conditions which promote neutral lipid accumulation [1]. Indeed, TLC analyses of cells overproducing FcrA indicated that they contained significant amounts of WEs under these conditions (FIG. 5A). Thus, overproduction of FcrA resulted in the appearance of a large band that ran similarly to stearyl stearate. Gravimetric analysis indicated that the FcrA-overproducing strain accumulated WEs to 13±5% of CDW under N⁻ conditions. This exceeds the levels of any natural or engineered strains reported to date, including A. calcoaceticus and M. aquaeolei VT8, which accumulated de novo WEs to ˜6% and ˜10% CDW, respectively [29, 51]. Similarly, engineered strains of E. coli and Acinetobacter accumulated WEs to 1% and 3% CDW, respectively [52, 53], or resulted in large reductions to growth yields [54].

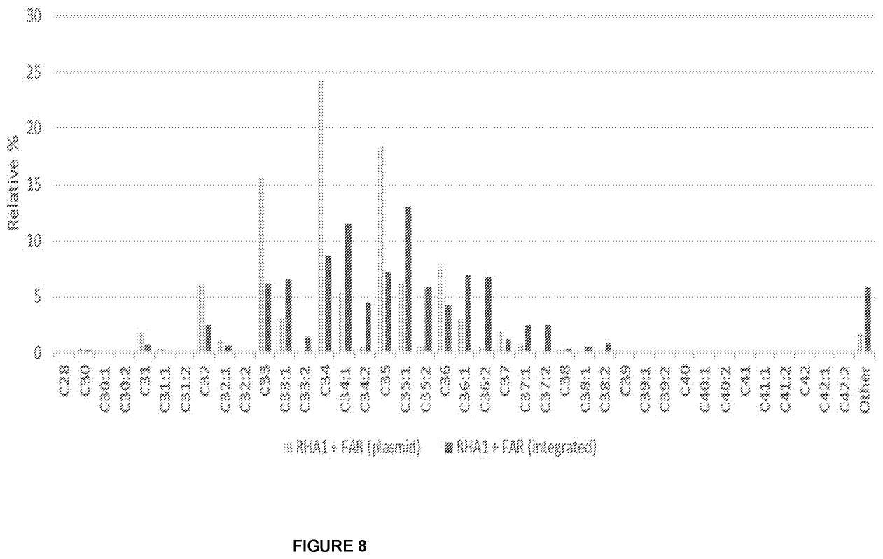

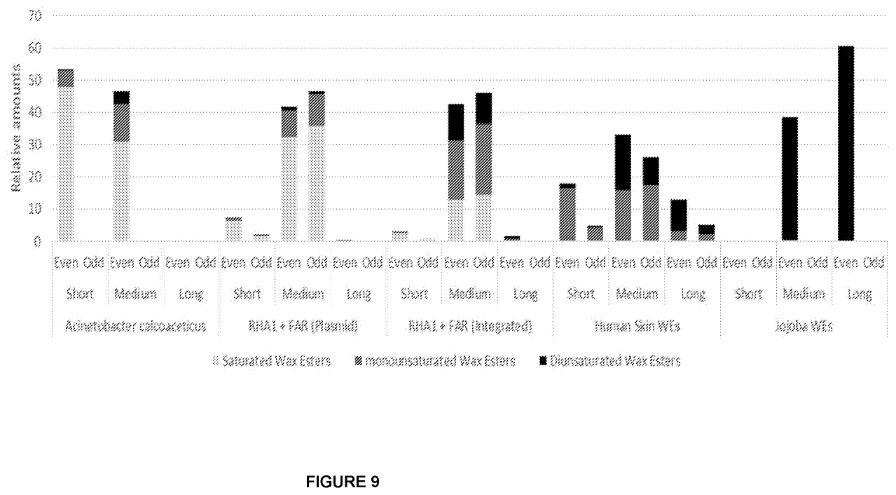

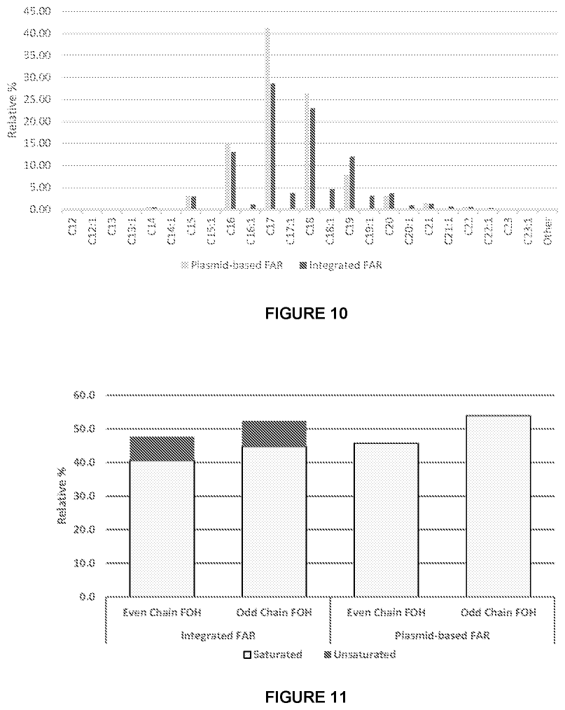

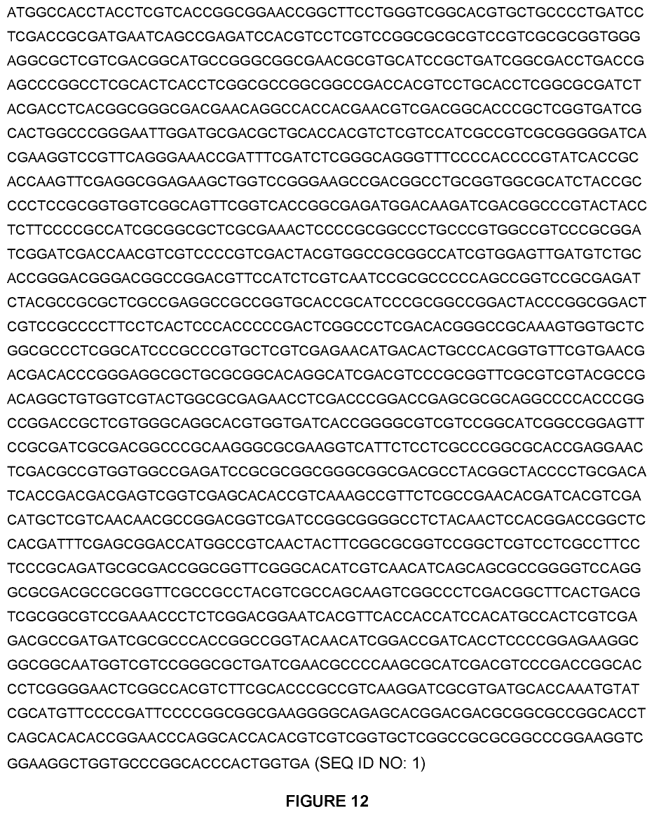

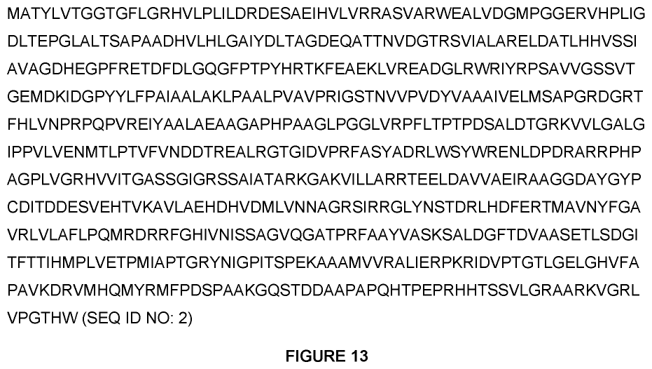

Produced WEs ranged from 30 to 38 C atoms in length (FIGS. 5B and 8). The WEs were on average a couple of carbon atoms longer than those in WT Cells (FIG. 3A). Moreover, a significant portion (˜20%) of the WEs were unsaturated. Analysis of the fragmentation ions further revealed that the most abundant saturated and unsaturated fatty acyl chain lengths were C17 (FIG. 3B). Finally, only trace amounts (<1% of total) of unsaturated fatty alcohols were detected, signifying that the unsaturation was primarily located within the acyl moiety (FIGS. 10 and 11). Therewith, the WEs accumulated by the engineered strain were similar to spermaceti WEs, which were historically valued as lubricants and cosmetics due to their excellent physicochemical properties [55]. Their similarities comprised several characteristics, including range of chain lengths, with C34 WEs being the majority species, lengths of acyl- and alcohol-components, and degree of unsaturation [27]. These composition characteristics also make the WEs of the engineered strain similar to WEs produced by sebaceous glands and deposited on the human skin [23, 24] (FIG. 9).

### Example 6: Wax Ester Accumulation in Rhodococcus opacus PD630

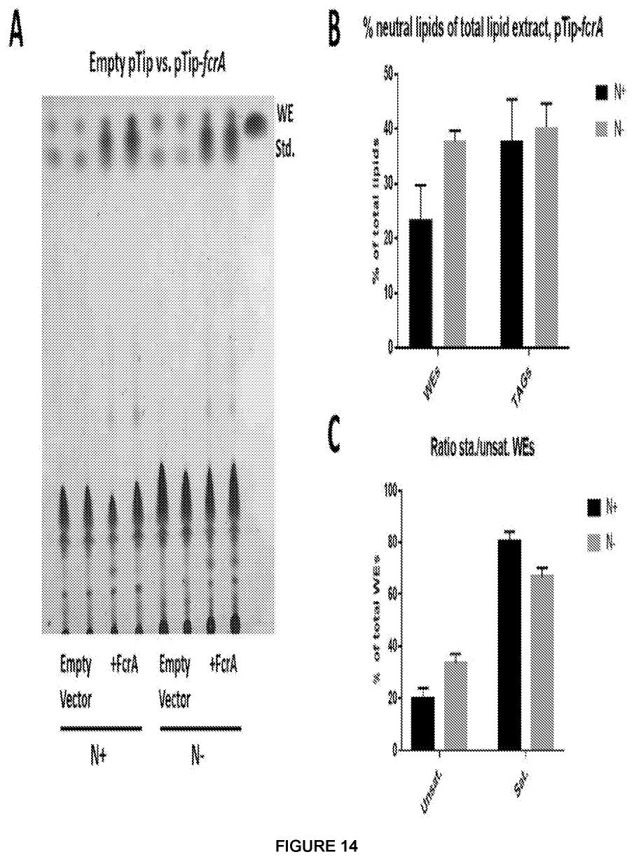

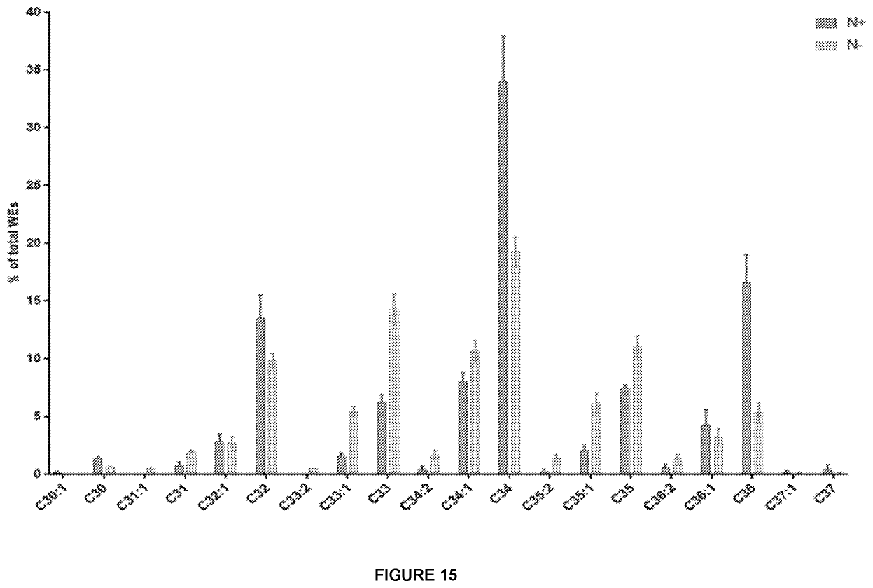

Wax ester (WE) accumulation was examined in Rhodococcus opacus PD630 (Herein PD630) by overproduction of FcrA. To test PD630's ability to accumulate WEs, the strain was transformed with pTip-fcrA and grown in carbon-limited (C−) and nitrogen-limited (N−) media. FcrA production was induced, and cells were harvested in exponential and stationary phases, for C− and N− cultures, respectively. WE accumulation was observed in both conditions (FIGS. 14A-C), and the produced WEs were similar to those of RHA1 (FIGS. 14A-C & 15).

### Example 7: Expression of fcrA Using Various Strength Constitutive Promoters

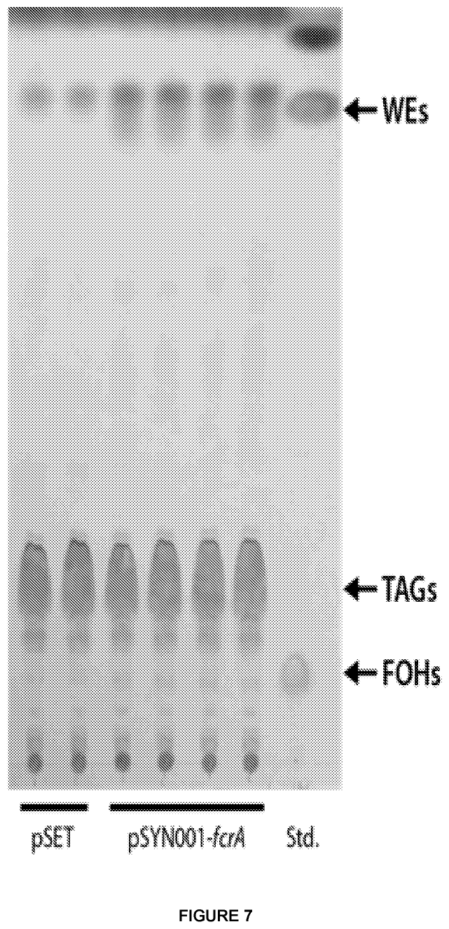

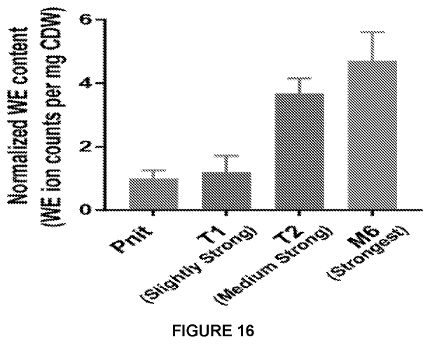

FcrA was integrated into the genome to create an industrially useful strain. Initially, the Pₙᵢₜ promoter was used to express a copy of fcrA integrated into the RHA1 genome (pSYN001-fcrA) resulting in the accumulation of WEs (FIGS. 7 and 8). To increase WE accumulation, fcrA was expressed with stronger constitutive promoters. Stronger constitutive promoters were identified. Promoters P_{M6} (strongest), P_{T1} (medium strong), and P_{T2} (slightly strong) were cloned into a pSYN vectors containing fcrA and transformed into RHA1. Transformed cells were grown in N− media, and WE accumulation was assessed. Increased expression of fcrA led to increased accumulation of WEs (FIGS. 16A-B). WEs produced by the integrated fcrA resulted in more unsaturated fatty alcohols then the plasmid-borne fcrA (FIGS. 10 and 11). As before, WEs of the engineered strain similar to WEs produced by sebaceous glands and deposited on the human skin [23, 24] (FIG. 9).

### Example 8: Co-Expression of fcrA and Wax Synthases

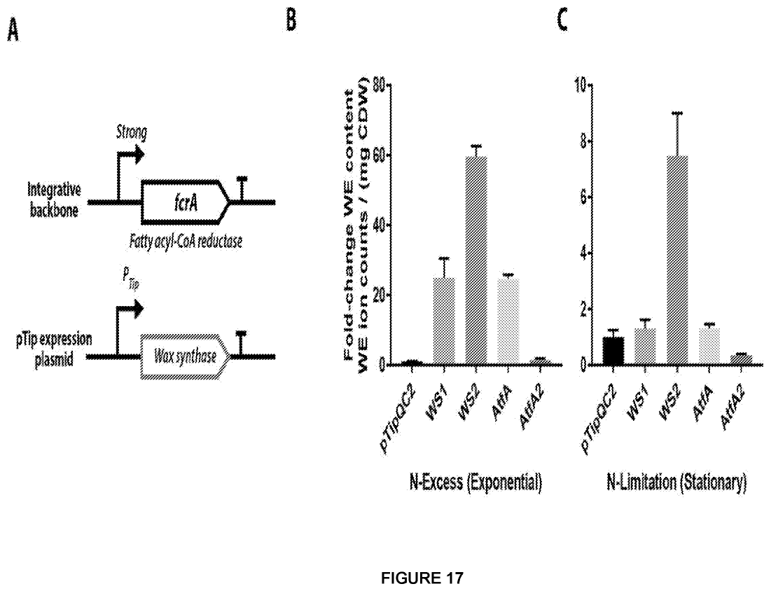

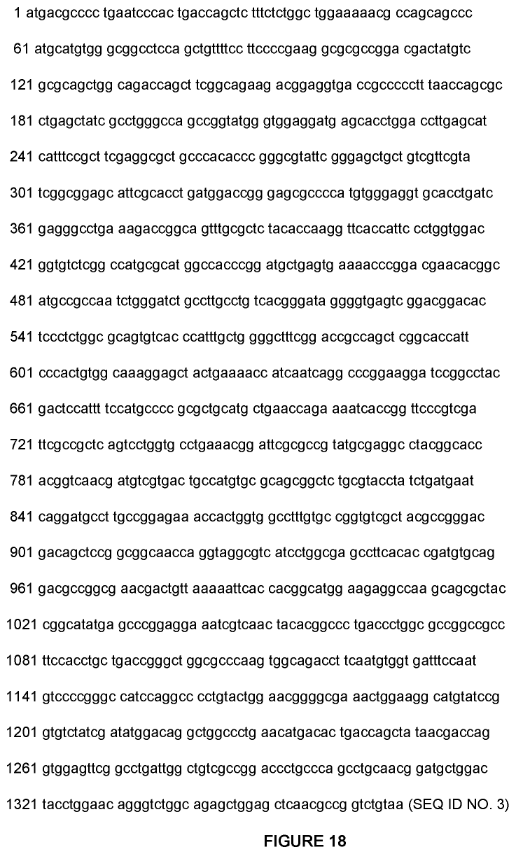

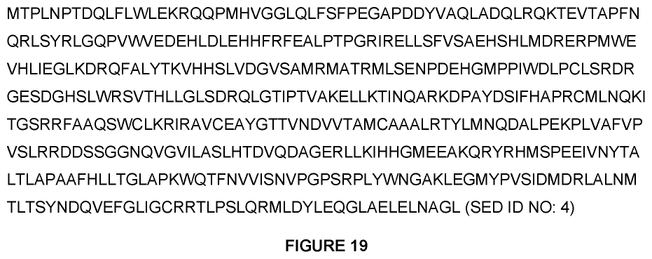

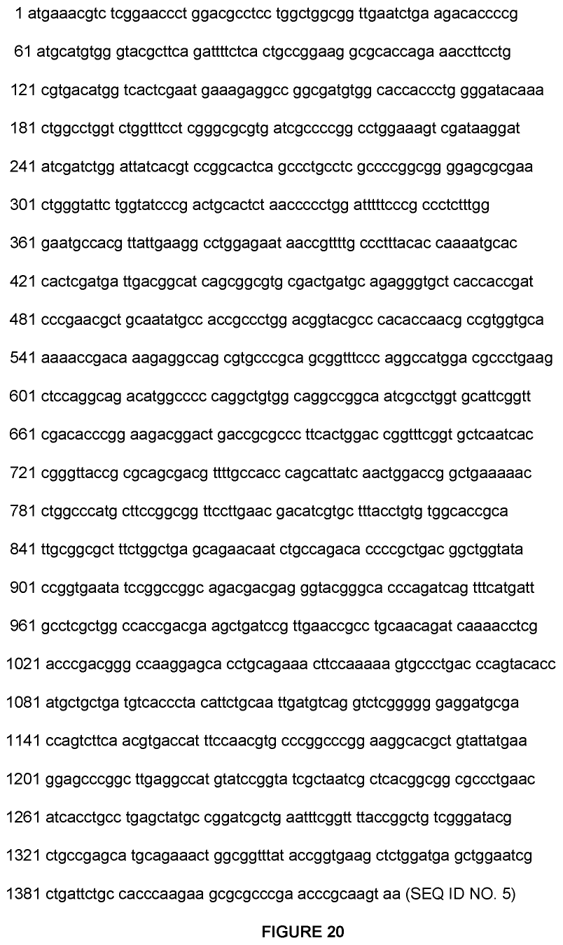

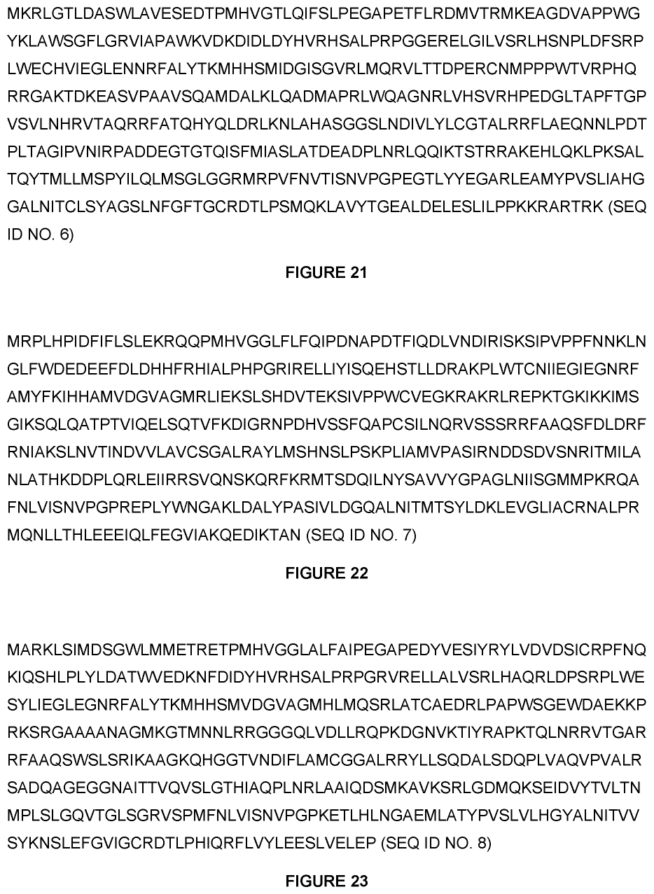

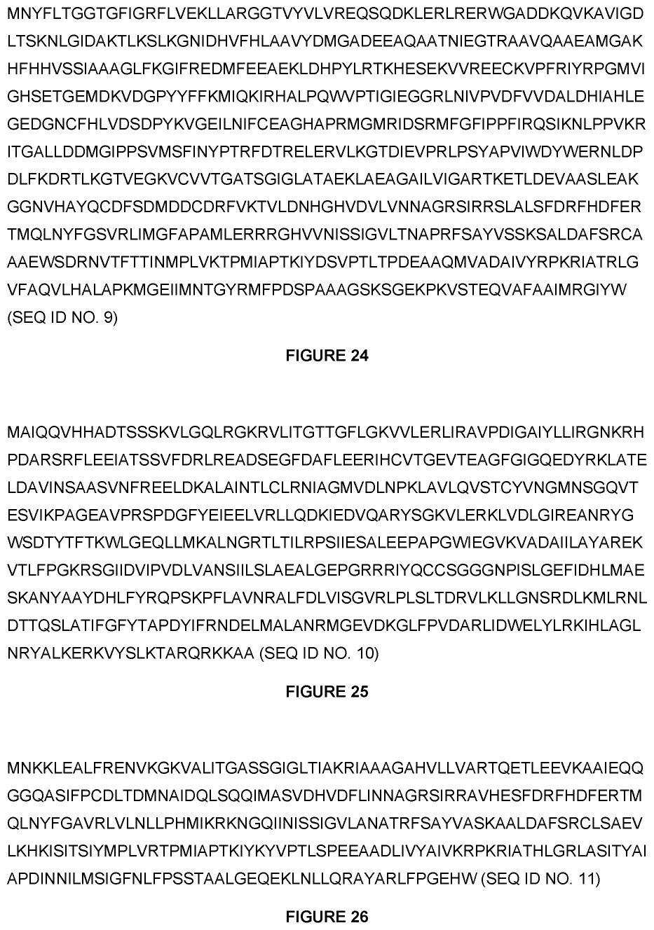

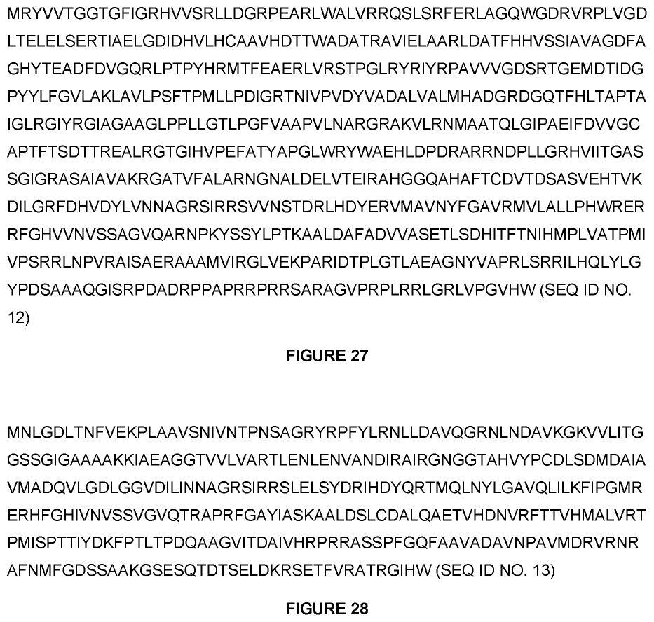

The co-expression of wax synthases (WSs) greatly increases WE accumulation. Previously characterized WSs (ws1, ws2, atfA, atfA2) were cloned into pTip vectors and co-transformed with pSYN004-fcrA into RHA1. Transformed cells were grown in C− and N− media. WSs expression was induced, and cells were harvested in exponential and stationary phases, for C− and N− cultures, respectively. Under C− conditions in exponential phase, expression of all WSs except affA2 led to an increase in WE content (FIGS. 17A-B). Under N− conditions in stationary phase, only the expression of ws2 led to an increase in WE content (FIGS. 17A-B).

### PATENT DOCUMENTS

Herreo, O. M.; Alvarez, H. M.: A process for industrial production of lipids from organic residues using different bacteria from Rhodococcus genus. WO 2016/075623.
Sinskey, J. A.; MacEachran, D.; Kurosawa, K.; Boccazzi, P.; Holder, J. W.: Production of triacyiglycerides, fatty acids and their derivatives. WO 2010/147642.

### OTHER PUBLICATIONS

1. Hernandez, M. A., et al., Biosynthesis of storage compounds by Rhodococcus jostii RHA1 and global identification of genes involved in their metabolism. BMC Genomics, 2008. 9: p. 600.
2. Dávila Costa, J. S., et al., Label-free and redox proteomic analyses of the triacylglycerol-accumulating Rhodococcus jostii RHA1. Microbiology, 2015. 161(3): p. 593-610.
3. Amara, S., et al., Characterization of key triacylglycerol biosynthesis processes in rhodococci. Sci Rep, 2016. 6: p. 24985.
4. Waltermann, M. and A. Steinbuchel, Neutral lipid bodies in prokaryotes: recent insights into structure, formation, and relationship to eukaryotic lipid depots. J Bacteriol, 2005. 187(11): p. 3607-19.
5. Ding, Y., et al., Identification of the major functional proteins of prokaryotic lipid droplets. J Lipid Res, 2012. 53(3): p. 399-411.
6. Liang, M.-H. and J.-G. Jiang, Advancing oleaginous microorganisms to produce lipid via metabolic engineering technology. Prog Lipid Res, 2013. 52(4): p. 395-408.
7. Alvarez, H. M., Biotechnological production and significance of triacylglycerols and wax esters, in Handbook of Hydrocarbon and Lipid Microbiology, K. N. Timmis, Editor. 2010, Springer Berlin Heidelberg: Berlin, Heidelberg. p. 2995-3002.
8. Steen, E. J., et al., Microbial production of fatty-acid-derived fuels and chemicals from plant biomass. Nature, 2010. 463(7280): p. 559-62.
9. Holder, J. W., et al., Comparative and functional genomics of Rhodococcus opacus PD630 for biofuels development. PLoS Genet, 2011. 7(9): p. e1002219.
10. Alvarez, H. M., et al., Identification of phenyldecanoic acid as a constituent of triacylglycerols and wax ester produced by Rhodococcus opacus PD630. Microbiology, 2002. 148(5): p. 1407-1412.
11. Vlysidis, A., et al., A techno-economic analysis of biodiesel biorefineries: Assessment of integrated designs for the co-production of fuels and chemicals. Energy, 2011. 36(8): p. 4671-4683.
12. Balan, V., Current challenges in commercially producing biofuels from lignocellulosic biomass. ISRN Biotechnol, 2014. 2014: p. 463074.
13. Keng, P. S., et al., Newly synthesized palm esters for cosmetics industry. Ind Crops Prod, 2009. 29(1): p. 37-44.
14. Jacobsen, E., et al., Age-related changes in sebaceous wax ester secretion rates in men and women. J Invest Dermatol, 1985. 85(5): p. 483-5.
15. Cooper, M. F., H. McGrath, and S. A. M. Shuster, Sebaceous lipogenesis in human skin. Br J Dermatol, 1976. 94(2): p. 165-172.
16. Grice, E. A. and J. A. Segre, The skin microbiome. Nat Rev Micro, 2011. 9(4): p. 244-253.
17. Schommer, N. N. and R. L. Gallo, Structure and function of the human skin microbiome. Trends Microbiol, 2013. 21(12): p. 660-668.
18. Brandwein, M., D. Steinberg, and S. Meshner, Microbial biofilms and the human skin microbiome. npj Biofilms and Microbiomes, 2016. 2(1): p. 3.
19. Zeeuwen, P. L., et al., Microbiome dynamics of human epidermis following skin barrier disruption. Genome Biology, 2012. 13(11): p. R101.
20. Kutlu, A. K., et al., A comparison study of growth factor expression following treatment with transcutaneous electrical nerve stimulation, saline solution, povidone-iodine, and lavender oil in wounds healing. Evid Based Complement Alternat Med, 2013. 2013: p. 9.
21. Nevin, K. G. and T. Rajamohan, Effect of topical application of virgin coconut oil on skin components and antioxidant status during dermal wound healing in young rats. Skin Pharmacol Physiol, 2010. 23(6): p. 290-297.
22. Mori, H.-M., et al., Wound healing potential of lavender oil by acceleration of granulation and wound contraction through induction of TGF-β in a rat model. BMC Complement Altern Med, 2016. 16: p. 144.
23. Picardo, M., et al., Sebaceous gland lipids. Dermatoendocrinol, 2009. 1(2): p. 68-71.
24. Tachi, M. and M. Iwamori, Mass spectrometric characterization of cholesterol esters and wax esters in epidermis of fetal, adult and keloidal human skin. Exp Dermatol, 2008. 17(4): p. 318-23.
25. Zouboulis, C. C., et al., Frontiers in sebaceous gland biology and pathology. Exp Dermatol, 2008. 17(6): p. 542-51.
26. Post-Beittenmiller, D., Biochemistry and molecular biology of wax production in plants. Annu Rev Plant Physiol Plant Mol Biol, 1996. 47: p. 405-430.
27. Nevenzel, J. C., Occurrence, function and biosynthesis of wax esters in marine organisms. Lipids, 1970. 5(3): p. 308-19.
28. Kaiser, J.-P. and K. W. Hanselmann, Fermentative metabolism of substituted monoaromatic compounds by a bacterial community from anaerobic sediments. Arch Microbiol, 1982. 133(3): p. 185-194.
29. Lenneman, E. M., et al., Fatty alcohols for wax esters in Marinobacter aquaeolei VT8: two optional routes in the wax biosynthesis pathway. Appl Environ Microbiol, 2013. 79(22): p. 7055-62.
30. Ishige, T., et al., Wax ester production by bacteria. Curr Opin Microbiol, 2003. 6(3): p. 244-250.
31. Kalscheuer, R. and A. Steinbuchel, A novel bifunctional wax ester synthase/acyl-CoA:diacylglycerol acyltransferase mediates wax ester and triacylglycerol biosynthesis in Acinetobacter calcoaceticus ADP1. J Biol Chem, 2003. 278(10): p. 8075-82.
32. Stoveken, T., et al., The wax ester synthase/acyl coenzyme A:diacylglycerol acyltransferase from Acinetobacter sp. strain ADP1: characterization of a novel type of acyltransferase. J Bacteriol, 2005. 187(4): p. 1369-76.
33. Rontani, J.-F., Production of wax esters by bacteria, in Handbook of Hydrocarbon and Lipid Microbiology, K. N. Timmis, Editor. 2010, Springer Berlin Heidelberg: Berlin, Heidelberg. p. 459-470.
34. Deb, C., et al., A novel in vitro multiple-stress dormancy model for Mycobacterium tuberculosis generates a lipid-loaded, drug-tolerant, dormant pathogen. PLoS ONE, 2009. 4(6): p. e6077.
35. Sirakova, T. D., et al., Wax ester synthesis is required for Mycobacterium tuberculosis to enter in vitro dormancy. PLoS One, 2012. 7(12): p. e51641.
36. Barney, B. M., et al., Differences in substrate specificities of five bacterial wax ester synthases. Appl Environ Microbiol, 2012. 78(16): p. 5734-45.
37. Alvarez, A. F., et al., Cloning and characterization of a gene involved in triacylglycerol biosynthesis and identification of additional homologous genes in the oleaginous bacterium Rhodococcus opacus PD630. Microbiology, 2008. 154(Pt 8): p. 2327-35.
38. Wu, G., et al., Metabolic Burden: Cornerstones in Synthetic Biology and Metabolic Engineering Applications. Trends Biotechnol, 2016. 34(8): p. 652-664.
39. Bauchop, T. and S. R. Elsden, The growth of micro-organisms in relation to their energy supply. J Gen Microbiol, 1960. 23(3): p. 457-69.
40. Sambrook, J. and D. W. Russell, Molecular cloning: A laboratory manual. 3rd ed. 2001, Cold Spring Harbor, N.Y.: Cold Spring Harbor Laboratory Press.
41. van der Geize, R., et al., Unmarked gene deletion mutagenesis of kstD, encoding 3-ketosteroid Δ¹-dehydrogenase, in Rhodococcus erythropolis SQ1 using sacB as counter-selectable marker. FEMS Microbiol Lett, 2001. 205(2): p. 197-202.
42. Willis, R. M., et al., Characterization of a fatty acyl-CoA reductase from Marinobacter aquaeolei VT8: a bacterial enzyme catalyzing the reduction of fatty acyl-CoA to fatty alcohol. Biochemistry, 2011. 50(48): p. 10550-8.
43. Bergmeyer, H. U., New values for the molar extinction coefficients of NADH and NADPH for the use in routine laboratories. Z Klin Chem Klin Biochem, 1975. 13(11): p. 507-8.
44. Eyer, P., et al., Molar absorption coefficients for the reduced Ellman reagent: reassessment. Anal Biochem, 2003. 312(2): p. 224-227.
45. Bligh, E. G. and W. J. Dyer, A rapid method of total lipid extraction and purification. Can J Biochem Physiol, 1959. 37(8): p. 911-7.
46. Pernet, F., C. J. Pelletier, and J. Milley, Comparison of three solid-phase extraction methods for fatty acid analysis of lipid fractions in tissues of marine bivalves. J Chromatogr A, 2006. 1137(2): p. 127-37.
47. Urbanová, K., et al., Structural characterization of wax esters by electron ionization mass spectrometry. J Lipid Res, 2012. 53(1): p. 204-213.
48. Creason, A. L., et al., Use of whole genome sequences to develop a molecular phylogenetic framework for Rhodococcus fascians and the Rhodococcus genus. Front Plant Sci, 2014. 5: p. 406.
49. Youngquist, J. T., et al., Production of medium chain length fatty alcohols from glucose in Escherichia coli. Metab Eng, 2013. 20: p. 177-86.
50. Aslan, S., et al., Wax esters of different compositions produced via engineering of leaf chloroplast metabolism in Nicotiana benthamiana. Metab Eng, 2014. 25: p. 103-112.
51. Fixter, L. M., et al., Structure, distribution and function of wax esters in Acinetobacter Calcoaceticus. J Gen Microbiol, 1986. 132: p. 3147-3157.
52. Kalscheuer, R., et al., Neutral lipid biosynthesis in engineered Escherichia coli: jojoba oil-like wax esters and fatty acid butyl esters. Appl Environ Microbiol, 2006. 72(2): p. 1373-9.
53. Santala, S., et al., Rewiring the wax ester production pathway of Acinetobacter baylyi ADP1. ACS Synth Biol, 2014. 3(3): p. 145-51.
54. Kannisto, M., et al., Growth and wax ester production of an Acinetobacter baylyi ADP1 mutant deficient in exopolysaccharide capsule synthesis. J Ind Microbiol Biotechnol, 2017. 44(1): p. 99-105.
55. Wolfmeier, U., et al., Waxes, in Ullmann's Encyclopedia of Industrial Chemistry. 2000, Wiley-VCH Verlag GmbH & Co. KGaA.
56. Shi, S. et al., Engineering of chromosomal wax ester synthase integrated Saccharomyces cerevisiae mutants for improved biosynthesis of fatty acid ethyl esters, Biotechnol. Bioeng. 2014; 111: 1740-1747.
57. Iven, T. et al., Synthesis of oleyl oleate wax esters in Arabidopsis thaliana and Camelina sativa seed oil. Plant Biotechnol J. 2016 January; 14(1):252-9. Epub 2015 Apr. 24.
58. Aslan, S. et al., Increased production of wax esters in transgenic tobacco plants by expression of a fatty acid reductase:wax synthase gene fusion. Transgenic Res. 2015 December; 24(6):945-53. Epub 2015 Jul. 3 All citations are hereby incorporated by reference.
59. Tatiana, D. et al., Wax Ester Synthesis is Required for Mycobacterium tuberculosis to Enter In Vitro Dormancy PLoS ONE 7(12): e51641 (2012)
60. Willis, R. M. et al., Characterization of a Fatty Acyl-CoA Reductase from Marinobacter aquaeolei VT8: A Bacterial Enzyme Catalyzing the Reduction of Fatty Acyl-CoA to Fatty Alcohol, Biochemistry, 2011, 50 (48), pp 10550-10558
61. Hofvander, P. et al., A prokaryotic acyl-CoA reductase performing reduction of fatty acyl-CoA to fatty alcohol, FEBS Letters 585 (2011) 1873-3468
62. S Reiser and C Somerville, Isolation of mutants of Acinetobacter calcoaceticus deficient in wax ester synthesis and complementation of one mutation with a gene encoding a fatty acyl coenzyme A reductase, J Bacteriol. 1997 May; 179(9): 2969-2975.

The present invention has been described with regard to one or more embodiments. However, it will be apparent to persons skilled in the art that a number of variations and modifications can be made without departing from the scope of the invention as defined in the claims. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing.

1. A method for producing a fatty alcohol, the method comprising:
i) providing a host cell comprising a recombinant fatty acyl-coenzyme A reductase;
ii) growing the host cell under conditions suitable for increased production of a fatty alcohol.

2. The method of claim 1 wherein the recombinant fatty acyl-coenzyme A reductase comprises a nucleic acid sequence substantially identical to SEQ ID NO. 1 or a fragment thereof or comprises an amino acid sequence substantially identical to SEQ ID NO. 2 or a fragment thereof.

3. (canceled)

4. A method for producing a fatty alcohol, the method comprising:
i) providing a host cell, wherein the host cell is a Rhodococcus cell; and
ii) growing the host cell under conditions suitable for increased production of a fatty alcohol.

5. The method of claim 1 wherein the host cell further comprises a wax synthase.

6. The method of claim 5 wherein the wax synthase is recombinant or heterologous.

7. The method of claim 5 wherein the wax synthase comprises a nucleic acid sequence substantially identical to SEQ ID NOs. 3 or 5 or a fragment thereof or an amino acid sequence substantially identical to SEQ ID NOs. 4 or 6 or a fragment thereof.

8. (canceled)

9. The method of claim 1 wherein the fatty alcohol is converted into a wax ester within the host cell.

10. The method of claim 1 further comprising isolating the fatty alcohol or wax ester.

11. A fatty alcohol or wax ester produced in accordance with the method of claim 1.

12. A vector comprising a polynucleotide comprising a sequence substantially identical to SEQ ID NO. 1 or a fragment thereof.

13. The vector of claim 12 further comprising a sequence substantially identical to SEQ ID NOs. 3 or 5 or a fragment thereof.

14. The vector of claim 12 or 13 operably linked to a promoter.

15. The vector of claim 14 wherein the promoter is a constitutive promoter.

16. A host cell comprising the vector of claim 13.

17. The host cell of claim 16 wherein the host cell overexpresses a fatty acyl-coenzyme A reductase.

18. The host cell of claim 16 wherein the host cell overexpresses a wax synthase.

19. The host cell of claim 16 wherein the host cell overproduces a fatty alcohol and/or overproduces a wax ester.

20. (canceled)

21. A kit comprising the vector of claim 12, in combination with instructions for producing a fatty alcohol or a wax ester.

22. A composition comprising a mixture of wax esters of twenty-eight to forty-five carbon atoms in length wherein at least one ester group is located at position C14, C15, C16, C17, C18, or C19 on the carbon chain.

23. The composition of claim 22 wherein the ester group of the wax esters is located at position C14 in at least 0.5% of the mixture, at position C15 in at least 2% of the mixture, at position C16 in at least 10% of the mixture, at position C17 in at least 25% of the mixture, at position C18 in at least 20% of the mixture, at position C19 in at least 10%, and at position C20 in at least 3% of the mixture.
